SEQUENCE LISTING (48 sequences)

SEQ ID NO: 1 - DNA, 1058 bp, Ashbya gossypii
  misc_feature: Oligo 72
gatctctctcctacggcgcaaaagaagattgccttgtacacggcccaaggttttgataaactacctatctgtatcgcgaaaactcagtactccctgtcccatgatgccacctttgaagaacgtcccctccgattttgtgttccccataagggatatcagggccttccattggagcgggctacctgtacgcgctagcggcagagattcaaactatccccggtctctccactaacgcaggctacatgaatgttgaagtcagtgatgacggcgagattgaaggtcttttctaggtctacgattacgtatgcttagccacccccgctcatatacataattatgttgaacaggcatgctcaggctgcgttccctgtagtgcacccgttagtgcgcatgggccccgaagccgttgcttttagaaccggcatgcgacacataccggagacattactttagcgtcgcagtgacagttatgtcccgagacgctggcatgcaatcgatgtcgagcctagtgagtcagaaacggagtcagtgtcttcagacacacaagcgaaatcagggtcagctttgactaaacctaaaaaccatagtgcattacgtccgactgtatcttcacataaagttagggaggagaatcaaagcaaggtagcataacaaagtatggatttgatggtcataaataaggacgactgggtatatgtacagtccgagggcgaccacagcggcgatcacacttattttgagagcacaatcacctcatcgcttcgagacacaaagcggcacgcgctattcccgcccatagtcagggagcagttcatccaggggaaagatatgtgcctaccaatctaccacgacgggataaaagagcaggcagcgctcgagtacgacggcacaggcgcgctatgcggcctttgacacgctgtggcaccacgccgccacacagcacgcaataaacgggcaacggtatattgctgatatacctcgatacttattattagaaaaaacctgcgtttgcaggcttttatactacataaagacaggatgaaaaacgtagatagatc

SEQ ID NO: 2 - protein, 48 aa, Ashbya gossypii
  misc_feature: Oligo 72
DLSPTAQKKIALYTAQGFDKLPICIAKTQYSLSHDATFEERPLRFCVP

SEQ ID NO: 3 - protein, 41 aa, Ashbya gossypii
  misc_feature: Oligo 72
SIGAGYLYALAAEIQTIPGLSTNAGYMNVEVSDDGEIEGLF

SEQ ID NO: 4 - DNA, 4474 bp, Ashbya gossypii
  CDS (440)..(3253)
  misc_feature: Oligo 72
cggcttcagttttttttatccccaattactctcgttactgattccgcgattgacgctaatttaactttagctttttatatgcttgacttagcctttacctgttgtcgactttctttagttcagcatcttgtatattacttgttcacgtataaatagatagcgatacccctttgcgagtatacagttgccttactgtgattatgttcttcctgttttatttgctaggtaagttttgacctttttttgatgttcacaagcaccgtcgacgctcaagaaaacgatcgaagttcctccctaacgtctgccgtttaccaagtataacgttgaactgtactatcctcctgtgcactaagttagcatgcttcgcacctttacggagtggagtattctaaggagtaatactcagctcggggctttcagctgcccagtacgaagacattaccatttaatttctggtaaaagcatcgcgaaaaccattagagacaatgctctgagagaggtgaaagagattaggcagcaagttccagattttaatccaaaactgaagatcttgcaggttggctcccgccctgattcatctgcatatgtccggatgaagttgaaagcatccaccgagtctggtgtagaatgtgacgttgaaaagcttccagaagatattacgcaactcgaactactcagaaagatcgagaaaatcaactctgacgccgaagtgcatgggattctgattcagctgccactgccaaagcatttagatgaagtgacagttaccaatgctgtggaccatgaaaaggacgttgatgggttccatcgctataatgttggtgagctcgctaaacgtggcgggaaaccatacttcctgccatgtacacctaatgcgtgtatacagcttttgaaagagtgcggtgttgagataagagggaaacaggcagtcgtaattggccgctctgacatcgtggggaccccagttgcaacgatgctcaaaaatcttgatgccacggtgactgttgcgcacagacatacgacaaatcttccacaagtcctctccactgcggatattgtagttgctgcgtgtggcgtgcctaactacataaagggcgaatggttaaaagatggcgccgtggttattgacgttggcatcaattatgttcaggattccaccaaaaaatcaggccaaagattagtcggtgatgtggacttcgaatcggcaaaggagaaggcttctcacatcacgcctgtacctggcggcgtgggtcctatgaccgtggccatgctggtgcaaaatgtcttgactgcagcaaaaagatcgttgcagaaggacgcagaggtgccagtgatcacaccactgcctgtgaattttgagatgcctgtgccatcggatatcgatatatcaaggaaacagaatccgaaacatatttctcaggtggcagccgagttgggtattagggatcacgagctggagttgtttgggcattataaagccaagatttctccaaaactcctccaaagattgcatgctaaccaaaatgcaaactatgtcttagttgctgggattacaccgacccctctaggagagggaaagtcgaccaccactatggggctagtccaagcgctttccgctcacctaggcaaacgggctattgcaaacgttcgtcaaccttcaatgggacccacgtttggagtcaagggaggcgcggcaggcgggggttacgcccaggtcattccaatggatgagttcaacttgcatttgacaggcgacatccacgccatttcagccgccaacaatctgctagccgcagccatagatacaaggatgtttcacgaacacacgcaaaagaacgatgctactttttataacagattggtgcccaagaagggcggctcgcggaagttcacgaaatcaatgctgaagaggctccagaagctgggaattaacaaaacagacccagacagtttgtctccaaaagaaatcaaacgatttgcacgcctaaacatagacacggataccatcactatcaaacgggtggttgatgtcaacgatagacttctacggcagattacgatcggccaagctcatactgagaagggttttaccagaaccaccgggtttgatatcactgttgcatctgaattgatggccattttggcactttctaaggacttcgctgatatgagagaacgtgtaggtcgcatggttgtcgctgcgaatctagacggcgagccgattacagcagaagatgtaggctgtgctggagctgttgctgcccttctgaaggacgctatcaagccaacactaatgcagtctttggaaggcacccctgttttagtccatgcagggccgttcgccaacatctctatcggagcctcctcagtaattgctgacagaattgccctgaagttggtgggcacttctccacattctccggaaggcaccgacgccggctatgtggttaccgaggccggattcgactttacaatgggcggagaacgattcctgaacataaagtgccgctcctcaggcttgaagccaaatactatcgtcatcgtggccactgtccgtgccctgaagctgcacggcggtggccctgaggtaaagccgggccagccacttactccccactatactgaggaaaacctcgagctagtcacaagaggtgcggccaacctgcagaagcaaataagaaatgccaagcttttcggggtccctgtcgtagtcgctgtgaacaggtttgagtcagacactgtcgcagaaattgagattattcagaaggcggcgaaggaggctggtgcacatgacgccatcgatgcaaatcgctgggccgaaggcggaaaaggagcagtcacgctggccgaagccgtcatcaactcatgcaaacaacccagttcttttaagttcctttatgacgtcgatcaacctgtgacagaaaagctctccaccattgttcggaaaatgtatggcggttccgcgatagatctctctcctacggcgcaaaagaagattgccttgtacacggcccaaggttttgataaactacctatctgtatcgcgaaaactcagtactccctgtcccatgatgccactttgaagaacgtcccctccgattttgtgttccccataagggatatcaggccttccattggagcgggctacctgtacgcgctagcggcagagattcaaactatccccggtctctccactaacgcaggctacatgaatgttgaagtcagtgatgacggcgagattgaaggtcttttctaggtctacgattacgtatgcttagccacccccgctcatatacataattatgttgaacaggcatgctcaggctgcgttccctgtagtgcacccgttagtgcgcatgggccccgaagccgttgcttttagaaccggcatgcgacacataccggagacattactttagcgtcgcagtgacagttatgtcccgagacgctggcatgcaatcgatgtcgagcctagtgagtcagaaacggagtcagtgtcttcagacacacaagcgaaatcagggtcagctttgactaaacctaaaaaccatagtgcattacgtccgactgtatcttcacataaagttagggaggagaatcaaagcaaggtagcataacaaagtatggatttgatggtcataaataaggacgactgggtatatgtacagtccgagggcgaccacagcggcgatcacacttattttgagagcacaatcacctcatcgcttcgagacacaaagcggcacgcgctattcccgcccatagtcagggagcagttcatccaggggaaagatatgtgcctaccaatctaccacgacgggataaaagagcaggcagcgctcgagtacgacggcacaggcgcgctatgcggcctttgacacgctgtggcaccacgccgccacacagcacgcaataaacgggcaacggtatattgctgatatacctcgatacttattattagaaaaaacctgcgtttgcaggcttttatactacataaagacaggatgaaaaacgtagatagatcagagtacgctatggtttcaggttgataatttatacaaaatgcgcgccattggaggaaccctccagtgcttaggcatcttcctttagcaaagcctctctcttttcagcgattctttgagctcttctctctctggcagctctgttcttcaatcttctggcctcagcctcctccttcaagggcttctcacgctgagcatcagccttggcctggataatgtgttcgaccaaggatctcttgtgcttgaaggtgttacccttggcagccttgtacaaagagtggtacaagtgtctgtcgatcttgccggcgtctctgtacttagccaacaatcttctcaagacacgcaatcttctgatccagacgacctgggatggcagtctaccctccttggtaccctttcctcttaccgtaaccgggggtgacggggcgttttcttggactcagccatggctc

SEQ ID NO: 5 - protein, 938 aa, Ashbya gossypii
  misc_feature: Oligo 72
YHLISGKSIAKTIRDNALREVKEIRQQVPDFNPKLKILQVGSRPDSSAYVRMKLKASTESGVECDVEKLPEDITQLELLRKIEKINSDAEVHGILIQLPLPKHLDEVTVTNAVDHEKDVDGFHRYNVGELAKRGGKPYFLPCTPNACIQLLKECGVEIRGKQAVVIGRSDIVGTPVATMLKNLDATVTVAHRHTTNLPQVLSTADIVVAACGVPNYIKGEWLKDGAVVIDVGINYVQDSTKKSGQRLVGDVDFESAKEKASHITPVPGGVGPMTVAMLVQNVLTAAKRSLQKDAEVPVITPLPVNFEMPVPSDIDISRKQNPKHISQVAAELGIRDHELELFGHYKAKISPKLLQRLHANQNANYVLVAGITPTPLGEGKSTTTMGLVQALSAHLGKRAIANVRQPSMGPTFGVKGGAAGGGYAQVIPMDEFNLHLTGDIHAISAANNLLAAAIDTRMFHEHTQKNDATFYNRLVPKKGGSRKFTKSMLKRLQKLGINKTDPDSLSPKEIKRFARLNIDTDTITIKRVVDVNDRLLRQITIGQAHTEKGFTRTTGFDITVASELMAILALSKDFADMRERVGRMVVAANLDGEPITAEDVGCAGAVAALLKDAIKPTLMQSLEGTPVLVHAGPFANISIGASSVIADRIALKLVGTSPHSPEGTDAGYVVTEAGFDFTMGGERFLNIKCRSSGLKPNTIVIVATVRALKLHGGGPEVKPGQPLTPHYTEENLELVTRGAANLQKQIRNAKLFGVPVVVAVNRFESDTVAEIEIIQKAAKEAGAHDAIDANRWAEGGKGAVTLAEAVINSCKQPSSFKFLYDVDQPVTEKLSTIVRKMYGGSAIDLSPTAQKKIALYTAQGFDKLPICIAKTQYSLSHDATLKNVPSDFVFPIRDIRPSIGAGYLYALAAEIQTIPGLSTNAGYMNVEVSDDGEIEGLF

SEQ ID NO: 6 - DNA, 918 bp, Ashbya gossypii
  misc_feature: Oligo 81
gatcgattcgctttcgtgagacacttggcgtaataccacacgcgagccgcaatgggccgccttaacgaggtagtgggcatatagtttctccttctgagttagcctgttcaaagaattttcgagcgctgagcataataacgggagcattccgttccgccaagtataactgactgagctggttgttcgtagtagccatcgttcttagaatgctaaatgtattacgcgaatgtatcgatactcgcttaaatatcgactgtagcatataaaaacgaacgaagcagttggtcctggtccttatttatgtacaagcattacgttcaaagttagtgaccaatgttgcaaaaagcctttttggaggatttcatagcgccgtttcctcggggggaggaagactactttaatttaggcaagtaaagtctgcgccagcctcaactgagtcacgccatcagatgacgacggatggatactcatctagccgcaagtattagtcgctgatttacgcgccgttctattgatctgattggctacttcggaatgtaaatagaatttacaaagtcagggattcacctttgtccttcttggattgaccgtactttttcatgcgaattgcttgaatagcgatgaaaccagtggtatcagtcggctggaatccggtcaattcgtccatggaggactccgtcgcatcatacaagttctcagtttccgaggacctgcccaacacaatgacattacccttgtaaagtctcaagcgaacctggccatttaccgtaatttgggatggcttaatcatgctacggatatactctgcattccggagaaaagtaggagccattgtagaggaggcgagagtaagtcggctgtaacaaattggtcacgcaaatgccgaacttctttgtccagggtgagaccctcgagatc

SEQ ID NO: 7 - protein, 20 aa, Ashbya gossypii
  misc_feature: Oligo 81
DLEGLTLDKEVRHLRDQFVT

SEQ ID NO: 8 - protein, 104 aa, Ashbya gossypii
  misc_feature: Oligo 81
YSRLTLASSTMAPTFLRNAEYIRSMIKPSQITVNGQVRLRLYKGNVIVLGRSSETENLYDATESSMDELTGFQPTDTTGFIAIQAIRMKKYGQSKKDKGESLTL

SEQ ID NO: 9 - DNA, 2898 bp, Ashbya gossypii
  CDS (716)..(1957)
  CDS (2246)..(2839)
  misc_feature: Oligo 81
gtgcagtacttcatctgcgtgagaaggggttcgggcgcacatggttggtaaatgaaatgccgtttgttcagaggcgtatcttctgtagtcaggaactcccgcccgtccaacgaatgggaaacgacaaattgcgggaacgcgggccttgccgcatcagagtttgtataaacaagatcgccctcctggtaagatataactggggcactcatcgcacactttacccacgcggcttgtactgatctctctgagcttgccgacgccctgaactttctgcctgttgcaagccatcacgtgacaaaatattacatatcacgtgttacaagcagaaccagtctgagtacggttatgcattggtccataatctgcgcgtgagggtccgcctgcccgctactttgtactcaatgtcccacttgtgcacttttctttcgtgtcctgcggcaggagtcacccgccccaatagttatgctgtcttagccgcctagctacgttggcgtacacacgtagcgagcaccaacagtttggtagatcaagccttacctagctctgtgactcaaccattcagtcatagttctgtttttctgctagtggtctggtagtgtactcttgcgagaatggctatgagtcactttttttgattcatgtgataagcttgttcggagtatataaacatcccttacgcgtttcgtttggaagatctctcacatccggaagcaagatggcacgcggaaaggtttgcttggcatactcaggaggcctggacacttcggtgatcctcgcctggttactggagcagggttatgaagttgtagcgtttatggctaatgttgggcaagaggaagatttccaggctgcggaagagaaggcattgaagataggagcgtccaagtttgtgtgcgttgactgccgtgagcagttcgtgacggatgtcttgtttcctgcggtgcaggtgaacgcggtgtacgaagatgtttatctactggggacgtcgcttgcacggcccgtgattgccaaggcacagatcgacatcgcggagcaggagcattgttttgcggtcgctcacggatgcactgggaagggcaacgatcagatccggtttgagcttgcgttctacgcgctgaagccagacgtccaggttatcgcgccatggcgggaccctgcgtttttcgaccggtttgcaggtagaaatgacttgctagagtgtgcggcagagaaacagatacctgttgcgcagaccaaggccaaaccgtggtctaccgacgagaacgaggcgcatatatcgtacgaggctggaattttggaggatcccgatgtcacaccaccggcagacatgtggaagctcacgacagacccgcttcgagcaccaagtgagcctgaggacgtgacgttggtgttcaaggagggtgtccccactcagatgatttacaaggatggagaggcgagcaaaccgttggatatttttcttgcggcttccaaagtggcgcgtagaaacggcgttggcagaatcgacatcgtggaaaaccgttacatcaacctgaagtcgcgtggctgctacgaacaggcaccactgacactcctgcgccgggcccacgtgtatctcgagggtctcaccctggacaaagaggttcggcatttgcgtgaccaatttgttacgccgacttactctcgcctcctctacaatggctcctacttttctccggaatgcgagtatatccgtagcatgattaagccatcccaaattacggtaaatggccaggttcgcttgagactttacaagggtaatgtcattgtgttgggcaggtcctcggaaactgagaacttgtatgatgcgacggagtcctccatggacgaattgaccggattccagccgactgataccactggtttcatcgctattcaagcaattcgcatgaaaaagtacggtcaatccaagaaggacaaaggtgaatccctgactttgtaaattctatttacattccgaagtagccaatcagatcaatagaacggcgcgtaaatcagcgactaatacttgcggctagatgagtatccatccgtcgtcatctgatggcgtgactcagttgaggctggcgcagactttacttgcctaaattaaagtagtcttcctccccccgaggaaacggcgctatgaaatcctccaaaaaggctttttgcaacattggtcactaactttgaacgtaatgcttgtacataaataaggaccaggaccaactgcttcgttcgtttttatatgctacagtcgatatttaagcgagtatcgatacattcgcgtaatacatttagcattctaagaacgatggctactacgaacaaccagctcagtcagttatacttggcggaacggaatgctcccgttattatgctcagcgctcgaaaattctttgaacagctaactcagaaggagaaactatatgcccactacctcgttaaggcggcccattgcggctcgcgtgtggtattacgccaagtgtctcacgaaagcgaatcgatctttgatatgttactaagtatccataaagtgatggccgggcgctaccatgaatcagaagagaatactctcttcttagagtatacttctcaattccttagcaacttggggaacttcaattcatttggtgacactaagtttattccgcgctgtacccaagggcacttcttttcccttttgaaacaagccggcctcgacaagaaagcgaagcctgccactaatggatgtccatttaaaacctataaagaattagtggagattggcgtctatgccgttgatgcccaaatctgcaatgctaagtttcccggatcttggtcgtacgacggcttattactttaactgcgttagtaaagccgacatgcagttggttgcagaaggaggtctttgccgccctc

SEQ ID NO: 10 - protein, 414 aa, Ashbya gossypii
  misc_feature: Oligo 81
MARGKVCLAYSGGLDTSVILAWLLEQGYEVVAFMANVGQEEDFQAAEEKALKIGASKFVCVDCREQFVTDVLFPAVQVNAVYEDVYLLGTSLARPVIAKAQIDIAEQEHCFAVAHGCTGKGNDQIRFELAFYALKPDVQVIAPWRDPAFFDRFAGRNDLLECAAEKQIPVAQTKAKPWSTDENEAHISYEAGILEDPDVTPPADMWKLTTDPLRAPSEPEDVTLVFKEGVPTQMIYKDGEASKPLDIFLAASKVARRNGVGRIDIVENRYINLKSRGCYEQAPLTLLRRAHVYLEGLTLDKEVRHLRDQFVTPTYSRLLYNGSYFSPECEYIRSMIKPSQITVNGQVRLRLYKGNVIVLGRSSETENLYDATESSMDELTGFQPTDTTGFIAIQAIRMKKYGQSKKDKGESLTL

SEQ ID NO: 11 - protein, 198 aa, Ashbya gossypii
  misc_feature: Oligo 81
MLQSIFKRVSIHSRNTFSILRTMATTNNQLSQLYLAERNAPVIMLSARKFFEQLTQKEKLYAHYLVKAAHCGSRVVLRQVSHESESIFDMLLSIHKVMAGRYHESEENTLFLEYTSQFLSNLGNFNSFGDTKFIPRCTQGHFFSLLKQAGLDKKAKPATNGCPFKTYKELVEIGVYAVDAQICNAKFPGSWSYDGLLL

SEQ ID NO: 12 - DNA, 139 bp, Ashbya gossypii
  misc_feature: Oligo 86
gatccggtcagtagccacaaacagcaacgtctcctgatcaacctcgtagatatcccgcaccttaccacgcgccactaacggcaggatgccgcccagctgtgtctgcactatgctcatttttgttcgccgcccccagatc

SEQ ID NO: 13 - protein, 39 aa, Ashbya gossypii
  misc_feature: Oligo 86
MSIVQTQLGGILPLVARGKVRDIYEVDQETLLFVATDRI

SEQ ID NO: 14 - DNA, 1671 bp, Ashbya gossypii
  CDS (587)..(1492)
  misc_feature: Oligo 86
taacccttgcccggttcgctgcgctgcttcgcgtgcttgctgttttcgtcaccgtttcgatctacgaaatgtgtgcaacaagccgaaaaagcaaaccacccgctccactgactatctgtttatgcttagacgccgcaaacttgtttctctttgggcctactctttgattcctggctcagagccttccctaagtaaggaatttcaactacttgtatatcaaagccttgctacaattgccttctacggcctcaattaccacgaatgcactaagcagctcagctgttccccttgttatgaaacgaaatcatcaacagctaacaaatgcctagctttactcctgtgacaagctgccttcacgtttctttttcaaccgttgacaatgacgattaagtaacaatgcgtctcgaggaatttgctcgaggtcgggtggggcagccgcgaaatgtagaatgcacggcagtatgactccaggcagcgcgagcttgactctggtctatggccaatttaacagccccatatagacaagatatatatagagagatagcagatagtcccatctggaacgatctgggggcggcgaacaaaaatgagcatagtgcagacacagctgggcggcatcctgccgttagtggcgcgtggtaaggtgcgggatatctacgaggttgatcaggagacgttgctgtttgtggctactgaccggatctcggcctatgatgtgatcatggcgaatgctatccccgacaagggtgtgctgttgacgcggctgtctgagttctggtttaaattcttggagaaagacgtccgcaaccacctccgggcgccgtgtgaggggctttttggtggtctacctgatgcgctaacgtcacccgagtaccgctcacagctagaggggcggagcctgctcgtgcaacggtaccggttgataccacttgaggtcatcgtgcgcggttacattacgggctctgcctggaaggagtaccaggagcgcggcacagtgcatggcctggctctacctgccgggctgcgtgagtctgaggagttacccgagccgatgttcactccgtctaccaaagcggagcagggtgcgcacgatgagaatatatcaccagcacaggctgcagacctaattgggcaggagctttgtgaccgcgtggctgccttggcaattcgcttgtattcgaagtgcaagaagtatgccagggagcgcggcattattattgcagacacgaagtttgagtttggcattgatgacaagtcaggcgagattgttcttgtcgatgaggtcttgaccccagattcttctcgtttctggaatgctgcgaactacgaagttggtaagcctcaggattcatatgataaacagtttttgcgcaattggctcactagcaacaagttgaatggtgtcgatggggtcagcatgcccgaggagatcgtgacgcgcacgaaggagaaatatgttgaggcctacgaagcacttacaggcgcgaaatggtaaaatgatgtacttttaaaatgttaaataattttaagtgataaatggagatgtgaaatttatgacctccatgggttagttggggtgcgcacatggtaccttgcatggatatctggtacgtcctgaatacgtttgccactcaccagctcccggggccgcttttctactgccaactcaaa

SEQ ID NO: 15 - protein, 302 aa, Ashbya gossypii
  misc_feature: Oligo 86
MSIVQTQLGGILPLVARGKVRDIYEVDQETLLFVATDRISAYDVIMANAIPDKGVLLTRLSEFWFKFLEKDVRNHLRAPCEGLFGGLPDALTSPEYRSQLEGRSLLVQRYRLIPLEVIVRGYITGSAWKEYQERGTVHGLALPAGLRESEELPEPMFTPSTKAEQGAHDENISPAQAADLIGQELCDRVAALAIRLYSKCKKYARERGIIIADTKFEFGIDDKSGEIVLVDEVLTPDSSRFWNAANYEVGKPQDSYDKQFLRNWLTSNKLNGVDGVSMPEEIVTRTKEKYVEAYEALTGAKW

SEQ ID NO: 16 - DNA, 1325 bp, Ashbya gossypii
  misc_feature: Oligo 162
gatctcgaactggcggatgatgcagtccaaggactacgctacatggacaaggccggggcacagcacatattgcacacgaagaatgtagttttctgtactggcgggtttgggtcgtccaaggagttgttggggaagtatgccccccatctccgggaccttcctacaacaaacgcgcagggtactacgggagatggacaggacctgatgctccgtgttggtgggcagctgattgatatggagcatatccaggtacaccccacgagttttgtggatcctaaagataaagacagcggtagcaaattcctggcggctgaggcactgcgcggcaatggaggtgtgcttttaaaccccagcactggccggcgctttacgaacgaattggcgacacgtgatgtacttacggctagcattcaagagcactgcaaagagcatgtggcttaccttgttctgagccaaggggcatatgaaactttgcaaagctttgtcaacttctacatctctaaagggctgatgcgagcgtccaccgttggcgagctttccgacgagatcggagttccgaaacaacagctggcgtcggagctagaggcgtactccgccgcagaggtggatcaattccagcgtcggcacattatcaatgattttggcccgagtgttgatgggtcgacagcaatatatgttggcttgatcacacccgcagtccattttaccatgggcggtgttcgaatcaattccagagcggaagtgctgagcagcgcagggacgacctataaagggctttacgccgctggagaggtatccggcggcgtgcacggtagaaacaggctgggtgggtctagtctcctggagtgcgtcgttttcgggaggcaggcagcaacatcgattacagaaaggttgcaggcgtgaactctagcaaccctggctcagtaacagacagtttttgcctgaaatgtgtcatttaaataacagcagatagtaggtagcatacaaaattaattagagacgtcaattaatactgcaattaggaaactagggatatcaactataattattaaaaacgtcgagtaaaaagaattaattggacggtccttgaaatagagccgaagaagctgaagaagaacagtaaatagtgaatacgatgagaaaggccttcaggtgccgcttttgagtgggtagacataaagcattttggtttcttagatcaatcgaaaggaatgtgcgtccttggatattttgatgctctttttaggtacaggtggactgccaaaaaatggcttgttcgctagcctaatgggtattattgcgtgaccaatctgccgtagtcagtt

SEQ ID NO: 17 - protein, 294 aa, Ashbya gossypii
  misc_feature: Oligo 162
DLELADDAVQGLRYMDKAGAQHILHTKNVVFCTGGFGSSKELLGKYAPHLRDLPTTNAQGTTGDGQDLMLRVGGQLIDMEHIQVHPTSFVDPKDKDSGSKFLAAEALRGNGGVLLNPSTGRRFTNELATRDVLTASIQEHCKEHVAYLVLSQGAYETLQSFVNFYISKGLMRASTVGELSDEIGVPKQQLASELEAYSAAEVDQFQRRHIINDFGPSVDGSTAIYVGLITPAVHFTMGGVRINSRAEVLSSAGTTYKGLYAAGEVSGGVHGRNRLGGSSLLECVVFGRQAATSI

SEQ ID NO: 18 - DNA, 2754 bp, Ashbya gossypii
  CDS (264)..(1772)
  misc_feature: Oligo 162
tcgtggaccatttgtcgcactggcgtgatgtctgtaaaaggaacggttaccgggatcacgccataccagctgctcccacggctcgaaaagtagtacaaataggtctatagcgacggtgtgcggcggcatctgctgatttcggaattcgctgtagtcaacggcgctggatacaggggcaccattcaggatacccagatttacaggaaagctagtcgtatggacaacttcgcccgtgcagcagtatatttatttgcatgtgaacaatgttaagagggcgtagcaagacagtgctgacattgatactgttacttgtaacggtcagactcctgtttaatcgcctacgttcgagaggcccagaaagcaatcggaggatagacatgtctggtaaagttcagcctgttgtcgtcgtcggactgggtcttgccggtctgtcgactggggcgcagctagtcaagaatggggtgccggtgatcttgatggacaaggtttctgccatcggcggaaattccgttaaggcctccagcggcataaacggcgatggtactcaggtgcaggagcttcttggtgtgtatgactctgctgactccttctacagagacaccgttgcgtcggcgaagggtgccggcgccagcgagctaatggacaagcttgcaagggactccgcaggtgctgtatcgtggctgcagaatgaatttggagttaaactggacatcctttcgcaactgggagggcattcgaaggctaggacacatcggtcgagcagcaacatacccccagggtttgagatcatatcggccatgcgcaagagactggagtcagtgcagaaggaaaacccagctctcgtgaacttccgcttggagagcaaagttgttgatctcgaactggcggatgatgcagtccaaggactacgctacatggacaaggccggggcacagcacatattgcacacgaagaatgtagttttctgtactggcgggtttgggtcgtccaaggagttgttggggaagtatgccccccatctccgggaccttcctacaacaaacgcgcagggtactacgggagatggacaggacctgatgctccgtgttggtgggcagctgattgatatggagcatatccaggtacaccccacgagttttgtggatcctaaagataaagacagcggtagcaaattcctggcggctgaggcactgcgcggcaatggaggtgtgcttttaaaccccagcactggccggcgctttacgaacgaattggcgacacgtgatgtacttacggctagcattcaagagcactgcaaagagcatgtggcttaccttgttctgagccaaggggcatatgaaactttgcaaagctttgtcaacttctacatctctaaagggctgatgcgagcgtccaccgttggcgagctttccgacgagatcggagttccgaaacaacagctggcgtcggagctagaggcgtactccgccgcagaggtggatcaattccagcgtcggcacattatcaatgattttggcccgagtgttgatgggtcgacagcaatatatgttggcttgatcacacccgcagtccattttaccatgggcggtgttcgaatcaattccagagcggaagtgctgagcagcgcagggacgacctataaagggctttacgccgctggagaggtatccggcggcgtgcacggtagaaacaggctgggtgggtctagtctcctggagtgcgtcgttttcgggaggcaggcagcaacatcgattacagaaaggttgcaggcgtgaactctagcaaccctggctcagtaacagacagtttttgcctgaaatgtgtcatttaaataacagcagatagtaggtagcatacaaaattaattagagacgtcaattaatactgcaattaggaaactagggatatcaactataattattaaaaacgtcgagtaaaaagaattaattggacggtccttgaaatagagccgaagaagctgaagaagaacagtaaatagtgaatacgatgagaaaggccttcaggtgccgcttttgagtgggtagacataaagcattttggtttcttagatcaatcgaaaggaatgtgcgtccttggatattttgatgctctttttaggtacaggtggactgccaaaaaatggcttgttcgctagcctaatgggtattattgcgtgaccaatctgccgtagtcagttagagggattcgtcatccacagtgttcttagttttcctaatcttgaaaaacttcacaccgctctggtgccgctgaggctctgcgacaggggcctgcaccgggcgcacgtcattgacgggcacagcgccgccctcaagctcggggttcgtgtcgtacacaatcgagtggtccatctggccatcgctcccgtgcttggtgccctggtacttgagcgcctgctccttgtagtgctgcatctcaacgtactccttgtgtgcggtccacgcgcggcggatgaaggagatcccgctgaagaagaagaccagcagcgagcacgccacactggcccacgcaatccccatcagctccggcccgatcttcgcctcgcgcccgtcgtcgcggaacacgttgcgcgccatcacaacaaccgccgtctggcacgccactgtcgctacgttgaacacgcacccgatcagcaccatggtgaaaccaccttggtgaacgagttcgagcaccacgtgaacacacagaacaaaaaacgcaattcgcaatgaaacgacagcgcgatccagaagagca

SEQ ID NO: 19 - protein, 503 aa, Ashbya gossypii
  misc_feature: Oligo 162
MLRGRSKTVLTLILLLVTVRLLFNRLRSRGPESNRRIDMSGKVQPVVVVGLGLAGLSTGAQLVKNGVPVILMDKVSAIGGNSVKASSGINGDGTQVQELLGVYDSADSFYRDTVASAKGAGASELMDKLARDSAGAVSWLQNEFGVKLDILSQLGGHSKARTHRSSSNIPPGFEIISAMRKRLESVQKENPALVNFRLESKVVDLELADDAVQGLRYMDKAGAQHILHTKNVVFCTGGFGSSKELLGKYAPHLRDLPTTNAQGTTGDGQDLMLRVGGQLIDMEHIQVHPTSFVDPKDKDSGSKFLAAEALRGNGGVLLNPSTGRRFTNELATRDVLTASIQEHCKEHVAYLVLSQGAYETLQSFVNFYISKGLMRASTVGELSDEIGVPKQQLASELEAYSAAEVDQFQRRHIINDFGPSVDGSTAIYVGLITPAVHFTMGGVRINSRAEVLSSAGTTYKGLYAAGEVSGGVHGRNRLGGSSLLECVVFGRQAATSITERLQA

SEQ ID NO: 20 - DNA, 1060 bp, Ashbya gossypii
  misc_feature: Oligo 178
gatcaatttccggtgctggtccgcagacaaggtcgtcttaccagtgccgctcaagccgaagaagagcgtcacatcgttgttggggccctggttggcggaggagtgcaatgtcaagacgttgtggttgataggcatcaagtagaacatcaccgtgaagatacccttcttcatctcaccagcgtactccgtgccaaggatgaccatctccatcgccctgaagttgatttcgacagtggtcttcgaggacataccctcagtgtgcgtgttcgctgggaactggcccgcgttccacacggtgaagtctggctcgccgaactccgcaagctcctcttcggtcggcctgatcaacatgttggtcatgaacaacgcgtggtatgcgcggcagcacacaatccggatcttgatgcggtagcgggggtcccagcccgcgtacgcatccacgatgtataggtgctcgcgggtacgcaagaagtctgcggcacgctcgcggttgatcaaccacgtcttctccgacgccttgcggttcactggaccccaccaaatattgtcagacgacgtgggctcgtccacgatccgcttgtccttaggcgaccgacccgtcttttccccagagtacgcaatcaacgcgccagtgctggagattgctgtcttgcgctccgctagcgcatcttcgtacaacaccgcggccggcgcattgcgccgaatcgtcaaaacgcccgagctgagcccaagctcagcgcggatcttctcctctggtgtttggtatacctttgatggcgacatagtgaacttattggcctgctagctctcctctgtgtagctgaagcttgtccacttgagacatgccatctactacgctagatgcctctatttatacacgccgaagcacaaggactgaaaattacgtctacttggctgtttgccgcagcgcccagtctctggccgttccactgtgttctacgcggcctgcaacagcgctgtcgctagcacaaactacgtcgcggacctatggatgaagcaagtacagcggtttcgctgtgctatccgtttttcgccgatc

SEQ ID NO: 21 - protein, 261 aa, Ashbya gossypii
  misc_feature: Oligo 178
MSPSKVYQTPEEKIRAELGLSSGVLTIRRNAPAAVLYEDALAERKTAISSTGALIAYSGEKTGRSPKDKRIVDEPTSSDNIWWGPVNRKASEKTWLINRERAADFLRTREHLYIVDAYAGWDPRYRIKIRIVCCRAYHALFMTNMLIRPTEEELAEFGEPDFTVWNAGQFPANTHTEGMSSKTTVEINFRAMEMVILGTEYAGEMKKGIFTVMFYLMPINHNVLTLHSSANQGPNNDVTLFFGLSGTGKTTLSADQHRKLI

SEQ ID NO: 22 - DNA, 2240 bp, Ashbya gossypii
  CDS (394)..(2016)
  misc_feature: Oligo 178
ccagcacaaaaaagagccgcgctgctccgggcagccgccggcagacggagccgagcacttaccgaaaggcctttccaacagctgctaaatgtgggggattgattcgcatgcttccggatcggcgaaaaacggatagcacagcgaaaccgctgtacttgcttcatccataggtccgcgacgtagtttgtgctagcgacagcgctgttgcaggccgcgtagaacacagtggaacggccagagactgggcgctgcggcaaacagccaagtagacgtaattttcagtccttgtgcttcggcgtgtataaatagaggcatctagcgtagtagatggcatgtctcaagtggacaagcttcagctacacagaggagagctagcaggccaataagttcactatgtcgccatcaaaggtataccaaacaccagaggagaagatccgcgctgagcttgggctcagctcgggcgttttgacgattcggcgcaatgcgccggccgcggtgttgtacgaagatgcgctagcggagcgcaagacagcaatctccagcactggcgcgttgattgcgtactctggggaaaagacgggtcggtcgcctaaggacaagcggatcgtggacgagcccacgtcgtctgacaatatttggtggggtccagtgaaccgcaaggcgtcggagaagacgtggttgatcaaccgcgagcgtgccgcagacttcttgcgtacccgcgagcacctatacatcgtggatgcgtacgcgggctgggacccccgctaccgcatcaagatccggattgtgtgctgccgcgcataccacgcgttgttcatgaccaacatgttgatcaggccgaccgaagaggagcttgcggagttcggcgagccagacttcaccgtgtggaacgcgggccagttcccagcgaacacgcacactgagggtatgtcctcgaagaccactgtcgaaatcaacttcagggcgatggagatggtcatccttggcacggagtacgctggtgagatgaagaagggtatcttcacggtgatgttctacttgatgcctatcaaccacaacgtcttgacattgcactcctccgccaaccagggccccaacagcgatgtgacgctcttcttcggcttgagcggcactggtaagacgaccttgtctgcggaccagcaccggaaattgatcggcgacgacgagcactgctggtctgactacggtgtgttcaacatagaaggtgggtgctacgccaagtgtatcggcctaagcggggagaaggagccggaaatcttcaatgcaatcaagtacggctctgttttggagaacgtggtctacgacccagtaacgcgtgtcgtggactatgaggactcttcgatcaccgagaacacacgttgcgcgtacccaattgagtacatcccaagcgcgcagattccatgcttgtccgagaaccatccttctaacatcgttctattgacctgcgatgcctcaggtgtgttgccacctgtgtcccgtttgacgccggagcaggtgatgtaccacttcatctcgggctacacctccaagatggccggtactgagcagggagtcactgagcctgaggctaccttctcttcctgcttcggccagccattcctagccctacatccaatgaaatacgctacgatgttggctgaaaagatgtcgcagcataacgctagcgcctggttgattaacacgggctggactggctcctcgtatacggccggcggtaagcgctgtccattgaagtacacccgtgctatcttggactcgatccatgacggaaccttggcccaggctgaatacgagaccctacccgtctttggtttgtccattccaaaagcggtcgagggcgtgccagctgagttgctaaaccctgccaagaactgggtcgagggcgaaggcaagtacgctagcgccgtgtcggctttggcctcgaagttcacagagaactttaagatctatcaggaccaggccacagaggaggttgtccgcgctggcccacaaatatgagatgcgaccatatagtggtttcagggatctgagtcttccggaccctatttataaggtaaaatactatatgtaataaataacatattcttgagaaactgcctgcctcatcggcgcggtgtcactaggcgcccgagggggccgtaaccacagtttgcagtcatgcggggtggtggcatcgtagtcgtagtggtgtgtgctgttccagcagcaggcttctcact

SEQ ID NO: 23 - protein, 541 aa, Ashbya gossypii
  misc_feature: Oligo 178
MSPSKVYQTPEEKIRAELGLSSGVLTIRRNAPAAVLYEDALAERKTAISSTGALIAYSGEKTGRSPKDKRIVDEPTSSDNIWWGPVNRKASEKTWLINRERAADFLRTREHLYIVDAYAGWDPRYRIKIRIVCCRAYHALFMTNMLIRPTEEELAEFGEPDFTVWNAGQFPANTHTEGMSSKTTVEINFRAMEMVILGTEYAGEMKKGIFTVMFYLMPINHNVLTLHSSANQGPNSDVTLFFGLSGTGKTTLSADQHRKLIGDDEHCWSDYGVFNIEGGCYAKCIGLSGEKEPEIFNAIKYGSVLENVVYDPVTRVVDYEDSSITENTRCAYPIEYIPSAQIPCLSENHPSNIVLLTCDASGVLPPVSRLTPEQVMYHFISGYTSKMAGTEQGVTEPEATFSSCFGQPFLALHPMKYATMLAEKMSQHNASAWLINTGWTGSSYTAGGKRCPLKYTRAILDSIHDGTLAQAEYETLPVFGLSIPKAVEGVPAELLNPAKNWVEGEGKYASAVSALASKFTENFKIYQDQATEEVVRAGPQI

SEQ ID NO: 24 - DNA, 1058 bp, Ashbya gossypii
  misc_feature: Oligo 64
tcaccaaaagggtgctcaagtttgtcctccggaaccggaagacagccttcgttagtttcatctccggctgggtggtttcaataacgtagtctttggacaatcaaacccgttaatctaccctggagcttcggtttatgaagaggagagagaagtctagcggcaaaagaagctcaatttaaaatagagatttgtgtttgccactacgatgttcagccgcgcattccagatacattcgtctggcacgatgaaataaccgccgaagtctcctcacgtgattattacgtaacatactttccgggtctcgaggatgcagtagtacaccccgaaataatgctacattagtcacgtggagatactgcagaagttgaagaagacaacggctttctccactactacatctacgaacagcaaggatcatgcaagctgttgatcggacgcagtttgcgccacttaagaatgacttgatgttaagggctgcgaggggtgaagatgttgaacgacctccgtgctggatcatgcggcaagcaggtcggtacttacccgaataccatgacgtgaagggtggccgtgacttctttgaaacctgccgtgatgctgaaatagcttcggaaatcacgcttcagcccgtaaggagatacgcaggccttttggacgctgcaattatcttctcggatattctggtgattccgcaggcgatggggatgcgcgtagagatgctggagggcaagggcccgcacttccccgagccactccggaccaccgagcaactgatgaaggtgctagagtacgaagtggatgtgctgcgtgagctcgactgggctttccgggccatcacactaacgcggcaaaagctggccggcgatgtgcctctcttcggcttctgtggagggccctggacattgctagtctacatgaccgagggaggtggctcgcgtctgttccgctttgccaaagaatggattcacaaacgtccagaacttgcgcataaactactgcagaaaatcactgacgttgcaattgagtttttggcgcagcaggttgtagctggttgccagatc

SEQ ID NO: 25 - protein, 206 aa, Ashbya gossypii
  misc_feature: Oligo 64
FAPLKNDLMLRAARGEDVERPPCWIMRQAGRYLPEYHDVKGGRDFFETCRDAEIASEITLQPVRRYAGLLDAAIIFSDILVIPQAMGMRVEMLEGKGPHFPEPLRTTEQLMKVLEYEVDVLRELDWAFRAITLTRQKLAGDVPLFGFCGGPWTLLVYMTEGGGSRLFRFAKEWIHKRPELAHKLLQKITDVAIEFLAQQVVAGCQI

SEQ ID NO: 26 - DNA, 1905 bp, Ashbya gossypii
  CDS (612)..(1712)
  misc_feature: Oligi 64
cctttaccctatccaacaaataatcgtgtaccttgtcatttgcatgcgacgcgtacggatgcgggtattgtgtgatgatctccaaatccaaccaggcatctgcaagcatctgcgcataattatagtctgatggcaatgtaaagtggtacctaaagtggtcccacacataaagagttgtgatcaccaaatatgtgatcaccaaaagggtgctcaagtttgtcctccggaaccggaagacagccttcgttagtttcatctccggctgggtggtttcaataacgtagtctttggacaatcaaacccgttaatctaccctggagcttcggtttatgaagaggagagagaagtctagcggcaaaagaagctcaatttaaaatagagatttgtgtttgccactacgatgttcagccgcgcattccagatacattcgtctggcacgatgaaataaccgccgaagtctcctcacgtgattattacgtaacatactttccgggtctcgaggatgcagtagtacaccccgaaataatgctacattagtcacgtggagatactgcagaagttgaagaagacaacggctttctccactactacatctacgaacagcaaggatcatgcaagctgttgatcggacgcagtttgcgccacttaagaatgacttgatgttaagggctgcgaggggtgaagatgttgaacgacctccgtgctggatcatgcggcaagcaggtcggtacttacccgaataccatgacgtgaagggtggccgtgacttctttgaaacctgccgtgatgctgaaatagcttcggaaatcacgcttcagcccgtaaggagatacgcaggccttttggacgctgcaattatcttctcggatattctggtgattccgcaggcgatggggatgcgcgtagagatgctggagggcaagggcccgcacttccccgagccactccggaccaccgagcaactgatgaaggtgctagagtacgaagtggatgtgctgcgtgagctcgactgggctttccgggccatcacactaacgcggcaaaagctggccggcgatgtgcctctcttcggcttctgtggagggccctggacattgctagtctacatgaccgagggaggtggctcgcgtctgttccgctttgccaaagaatggattcacaaacgtccagaacttgcgcataaactactgcagaaaatcactgacgttgcaattgagtttttggcgcagcaggttgaagctggttgccagatcctgcagttgttcgaaagttggggtggcgagctgtcccttgccgacttggacgaattttcgctgccgtacttgaagcaaattgcggctgccgttccggcacgtttacgcgaattaggcattactgaagatgtcccaatgacggtttttgccaagggctcgtggtatgctctagacaagctctgcgacgcaggctataacaccgtttccctcgactggctgtgggatcctgccgaggccgttcgtatcaacaacggcagagtcaccctacagggcaatctggacccgaccgtcatgtatggttccgatgagactatcaccaagaagactgaggcaatgatcaaaggattcggtggtggcaaaaaacagtacatcgtcaatttcggtcacggaacgcaccctttcatggacccagagaagatacgccattttcttgaggaatgccacagaatcgggaagatgtaaccttctggaagtagaagagcacattatcaggtcaatagcatcattatgtattattttagacgctagttgaactccaatatttttgatcatgtgagcgtatgacgggccactttaccacgcctagcaaaaatgttcgcggttgttctaccgatcataaaccacttcaccgttcggagaagcacttttgatc

SEQ ID NO: 27 - protein, 367 aa, Ashbya gossypii
  misc_feature: Oligi 64
MQAVDRTQFAPLKNDLMLRAARGEDVERPPCWIMRQAGRYLPEYHDVKGGRDFFETCRDAEIASEITLQPVRRYAGLLDAAIIFSDILVIPQAMGMRVEMLEGKGPHFPEPLRTTEQLMKVLEYEVDVLRELDWAFRAITLTRQKLAGDVPLFGFCGGPWTLLVYMTEGGGSRLFRFAKEWIHKRPELAHKLLQKITDVAIEFLAQQVEAGCQILQLFESWGGELSLADLDEFSLPYLKQIAAAVPARLRELGITEDVPMTVFAKGSWYALDKLCDAGYNTVSLDWLWDPAEAVRINNGRVTLQGNLDPTVMYGSDETITKKTEAMIKGFGGGKKQYIVNFGHGTHPFMDPEKIRHFLEECHRIGKM

SEQ ID NO: 28 - DNA, 972 bp, Ashbya gossypii
  misc_feature: Oligo 125
atctgctgcggccggatgtcaacatccaggagcttggaaatctgcgtcgtgcgctgggcctctagtttcccgccgccgttagtgagcaatatgaagggtatgcagttggcaggtccagcagcctgagcgcctcggccgcacccggaatgggcttttcgccgtggaggaggacgccgtcgatgtcaaacacaaacccgacgtctgtagagaggcgcctgatacgctgtaggcacttgagtggcgagaagcggagcattgctgtgctgggcgagctgccttcgcgaatggggtctgtgttccgtcggcaactgcgttcgcctgcgaggcggaattttattgtgtgtcatatctcgccggcgtgagctcagcctccgtcagcgacgtgcggcagagccacttggcgcgtgaactgggagcgctatttactatacaggaacaagtgtttctagtagtgtcgggatgtcatctccggtatccctcttcgacagagtagtgcagggcgcctgcctgttcggaccctaacccttttagtgaagtcactgcgtgaccgacaatcagcagccctggcgggcgtgagccgatctgttcgacgacctcaggcacgcttgcaagcgtggttctggtcacgcgctggtccggacacgaggcacgctcgacaatggctgctggcacctgagggtcccattgctgttccagcagggcctggacaaggtcccccacacgatgcagcgccattaggaaaattgtagtgcgtgcggcgacaaactcgggagcagggggcagcactccccgcctaccaggtgcctggtacatatcaagacctgctctgcgacgtcactgctgcgtggccggaatatgtgcgacagtagtggcagcgagagctgagctaataccaggtacaacgcgaggctcaaacccatgccgcttgaaaagcaggtactcttcgccgcctctaccgaagatgtacggatc

SEQ ID NO: 29 - protein, 147 aa, Ashbya gossypii
  misc_feature: Oligo 125
DPYIFGRGGEEYLLFKRHGFEPRVVPGISSALAATTVAHIPATQQRRRAGLDMYQAPGRRGVLPPAPEFVAARTTIFLMALHRVGDLVQALLEQQWDPQVPAAIVERASCPDQRVTRTTLASVPEVVEQIGSRPPGLLIVGHAVTSL

SEQ ID NO: 30 - DNA, 2455 bp, Ashbya gossypii
  CDS (275)..(2023)
  misc_feature: Oligo 125
ggtcgcaatggaataataaggttgtatatagttattctgccccacttcgatgccctgaggttttcggcgatgagcagtaaaaaagctcgatcaggccatcagcacgtgactagtacaaagccacagctgtggggttggtcaagcttatgatcagcgccgctcaaggatgaatcacaagcgactgtggccagatcctataaagagatatatacgcccgggaagatggagggcgttttgtggggtccatgctgtgattctttaaagggtgacatcgatgggccgtgaaggagtgccgctaattgcgtctttgaacgctagcaatgaagtcatgctactggtggggacaggcggatcagggcgagtggtcagtcgactgagggggttgcttaatacaggtgcacagtgcattgtggtacagccctcgaacagcaaactcgtggcgcaactatatcgggaatttgcgggggagccgctgctccggatcttagatcgggcgtttgcattggaagacctgacgaccctagggaggtacgaggccagcaatgtggtggatcgggtatttctgacgccgggtcacagtgctgatgagatattacaggcccatgcggtgtaccggcagtgcatcaaactgcgcattcccatcaacacgagccaacggccggaactttcgaccttttcgctgccgtcaacctacagggacccgaaaggctccgggttgcaggtggccgtcactactgatggccgcggctgcgtactggccaacagaatcagacgtgaaattgtgagcaagctgcctgccaatatctccgaggtagtcagcaacatgggtcggctgcgggactgcataaccacacgtgaccatgcggaattgctccagacggttgacttagagtccgagctcgagaccatgggcatcctgcccgatgaggaccaatgggcgtcgcaccgctttaataagctggtccaggagtttcggatgaaggagtccgaccagcgactcaagcggctccggtggctctcccagataatggagtactaccctttgagcgagctagcggatgtgtccgttgacaaactgttggatgagcagactggagccgcgtgttctggaacgacagcgacaaccggagccgcatccgtgactggggccacagaggacacggctgctgagcctgctgagggggtggcatccgatagctctggcgctcgcgacctagcacgcagtgccgcaggctccatcgcacttgttggcagtggccctggctccctctccatgctcacgctaggcgcactcagcgagattaagtccgcagaccttgtcctcgcagacaagctggttccgcaagaggtgatgaatacaattccagcgggcactgagaccttcatcgccagaaagttcccgggcaatgcggagcgcgcacaggaagagctagccgaaaaggctctagccgcgcttcgggagggaaagagggttgtgcgcttgaaacagggcgatccgtacatcttcggtagaggcggcgaagagtacctgcttttcaagcggcatgggtttgagcctcgcgttgtacctggtattagctcagctctcgctgccactactgtcgcacatattccggccacgcagcgtgacgtcgcagagcaggtcttgatatgtaccggcactggtaggcggggagtgctgccccctgctcccgagtttgtcgccgcacgcactacaattttcctaatggcgctgcatcgtgtgggggaccttgtccaggccctgctggaacagcaatgggaccctcaggtgccagcagccattgtcgagcgtgcctcgtgtccggaccagcgcgtgaccagaaccacgcttgcaagcgtgcctgaggtcgtcgaacagatcggctcacgcccgccagggctgctgattgtcggtcacgcagtgacttcactaaaagggttagggtccgaacaggcaggcgccctgcactactctgtcgaagagggatacggagatgacatcccgacactactagaaacacttgttcctgtatagtaaatagcgctcccagttcacgcgccaagtggctctgccgcacgtcgctgacggaggctgagctcacgccggcgagatatgacacacaataaaattccgcctcgcaggcgaacgcagttgccgacggaacacagaccccattcgcgaaggcagctcgcccagcacagcaatgctccgcttctcgccactcaagtgcctacagcgtatcaggcgcctctctacagacgtcgggtttgtgtttgacatcgacggcgtcctcctccacggcgaaaagcccattccgggtgcggccgaggcgctcaggctgctggaccgccaacgcatacccttcatattgctcactaacggcggcgggaaactagaggcccagcgcacgacgcagatttccaagctcctggatgttgacatccggccgcagca

SEQ ID NO: 31 - protein, 583 aa, Ashbya gossypii
  misc_feature: Oligo 125
MGREGVPLIASLNASNEVMLLVGTGGSGRVVSRLRGLLNTGAQCIVVQPSNSKLVAQLYREFAGEPLLRILDRAFALEDLTTLGRYEASNVVDRVFLTPGHSADEILQAHAVYRQCIKLRIPINTSQRPELSTFSLPSTYRDPKGSGLQVAVTTDGRGCVLANRIRREIVSKLPANISEVVSNMGRLRDCITTRDHAELLQTVDLESELETMGILPDEDQWASHRFNKLVQEFRMKESDQRLKRLRWLSQIMEYYPLSELADVSVDKLLDEQTGAACSGTTATTGAASVTGATEDTAAEPAEGVASDSSGARDLARSAAGSIALVGSGPGSLSMLTLGALSEIKSADLVLADKLVPQEVMNTIPAGTETFIARKFPGNAERAQEELAEKALAALREGKRVVRLKQGDPYIFGRGGEEYLLFKRHGFEPRVVPGISSALAATTVAHIPATQRDVAEQVLICTGTGRRGVLPPAPEFVAARTTIFLMALHRVGDLVQALLEQQWDPQVPAAIVERASCPDQRVTRTTLASVPEVVEQIGSRPPGLLIVGHAVTSLKGLGSEQAGALHYSVEEGYGDDIPTLLETL

SEQ ID NO: 32 - DNA, 447 bp, Ashbya gossypii
  misc_feature: Oligo 107
gccgcgatataattaagaaacggttggaatcggcagaactagaggttgaagagataatcacagtatcagtaccagcccacctccatgatattagtgagcagcctttgaacgtgtgttcaccgataactcttgggtcgtgctatttagtcctcagggcaccaatgaactgcttcccctactgagacagggggctgccaagattgcagctattggaccaacaactgcgcaatatctacttgacaatgccactccaccaatattaacgagccctaaacctgagccctcgagcttgtacaatgcaataaaagattatttggcagcgtctcctatgggcaattagttggagggcactatgtatgtatagctttatacttatgaaatggtgcagaacacgcatgtgttcacgttaccagtttatggagtctatgatcaaataacaaatatttt

SEQ ID NO: 33 - protein, 69 aa, Ashbya gossypii
  misc_feature: Oligo 107
ERVFTDNSWVVLFSPQGTNELLPLLRQGAAKIAAIGPTTAQYLLDNATPPILTSPKPEPSSLYNAIKDY

SEQ ID NO: 34 - DNA, 1626 bp, Ashbya gossypii
  CDS (250)..(1035)
  misc_feature: Oligo 107
tcacccttgggatccgctgactggctactgtaattctatgtagtggattagtacgataagttacttttgtatgatagatgtaatcacatctggctattaaaatgactcagccgaggtaaatctaacgtcccttcacaagggtgttcctgtgtggacttccgcctgaatttttatagatatatagatactctactcatgaacaacctgcaaccgaataagcattagtgccaggagaagagaaccgtggaaatggggcaagtagaaaaaatcatattccttaagaataagacagtaccagaggaccattacgagacgatttttgaatcgaatggcttccagactcactttgtacccataataacccatgaacacctgccagatgaggttcgcggtcgactatccgacgcgaattacatgaaaaggttgaattgtttggtggtaacctctcagaggactgtggagtgtctctatgaggacgttctgccctctcttccagctgaagcacgcaaatctcttctcaatacgccagtattcgtggttgggcgtgccactcaggaatttatggagagatgcggctttacggacgtgagagggggatctgagactggtaatggcgttttgctagcggagttaatgttaaatatgatccagaagggcgatatcagctcctcgggaacttttctgctgctcgtgggcgagatccgccgcgatataattaagaaacggttggaatcggcagaactagaggttgaagagataatcacgtatcgtaccagcccactccatgatattagtgagcgctttgaacgtgtgttcaccgataactcttgggtcgtgctatttagtcctcagggcaccaatgaactgcttcccctactgagacagggggctgccaagattgcagctattggaccaacaactgcgcaatatctacttgacaatgccactccaccaatattaacgagccctaaacctgagccctcgagcttgtacaatgcaataaaagattatttggcagcgtctcctatgggcaattagttggagggcactatgtatgtatagctttatacttatgaaatggtgcagaacacgcatgtgttcacgttaccagtttatggagtctatgatcaaataacaaatattttatcgcgctggtatatgtgcacaacttccaccactttgtccttctcgttgctatagatctctgcctttggatatattaaatagccgcgccgcaaagagatcaactgaatcgagaatactaaatggtcgcctactattacagaacggcgcctgccgttaattgaccagtcagggctgttgctgaactcgacaatatatgtcaatggttccctttccatcacccactgcgtagacaagttgcacacttctacatcaattgtgaccgcatcccccacatgtaatacttcgtggcttgtggcagacaagcgaacactgaatagctgtttgatggatggaagatgcactgggacaattaacgtattgggttggatactaaaccgtgtttgtcgggagagaactctatgccagaagctaataacttgagatagctaccaaaccgggtcaagatctcagaatcactaatgggacgaggagaggtcattc

SEQ ID NO: 35 - protein, 262 aa, Ashbya gossypii
  misc_feature: Oligo 107
MGQVEKIIFLKNKTVPEDHYETIFESNGFQTHFVPIITHEHLPDEVRGRLSDANYMKRLNCLVVTSQRTVECLYEDVLPSLPAEARKSLLNTPVFVVGRATQEFMERCGFTDVRGGSETGNGVLLAELMLNMIQKGDISSSGTFLLLVGEIRRDIIKKRLESAELEVEEIITYRTSPLHDISERFERVFTDNSWVVLFSPQGTNELLPLLRQGAAKIAAIGPTTAQYLLDNATPPILTSPKPEPSSLYNAIKDYLAASPMGN

SEQ ID NO: 36 - DNA, 368 bp, Ashbya gossypii
  misc_feature: Oligo 136
gatctcccatgtgtccaccggtggtggtgcctccctagagctattggagggtaaggacctaccaggtgtcaccttcttgtccagcaagcagtagatgctgtccgcgcgcctgccggccttgaatagataataaactatatacgcacgtaatggaaagttgagaaactactatatgcgcctgttgcgctgctcgtccgtgggctccagccgcgggaacccgagcagctccatgatgcggtactcgtccgcgctctcgacgcacgcgccgctcgacttgtcaaacaggccgtgctgattcagcgtcatgccctgcgcggaggccccgcgtccgcagagccttgttgaaaagtgtcgttgccgatgaaagt

SEQ ID NO: 37 - protein, 30 aa, Ashbya gossypii
  misc_feature: Oligo 136
ISHVSTGGGASLELLEGKDLPGVTFLSSKQ

SEQ ID NO: 38 - DNA, 2000 bp, Ashbya gossypii
  CDS (447)..(1790)
  misc_feature: Oligo 136
ctgcagcacgtggtcctgccggtttcgcagttcgccgcacagctcccgcaccgccatgtaccgaagatctacatcctcctgctcgcgatagcgcatcacgcaccgcagcgcgctctccatctccgtctccatccttgcgcttgcccgtatctgccaacaagccgtccagaacgccgctgcttagtacgagctcgtcacgtgcgactgccagcttcctgttctgtggaagccggcttctcccctggctttttccgtagcatgacgccggggctcaccagagaacgaggaagaatttccagttggcgagcacaggccaggcgggctccctacgggtaccgtggggcgagcaaggcagggcaatgatgcaataagcgcaggccggcacgcaattgagcgagcaacgtgcacgaggatttttctgggggcgaaaaaagcgagtataaaggatgggccggtcgtggggtctgctctggatggcgtgcttgctgtacggcgtgttcacagagtgtcgcagatcttcacagcacgcacacacaggaaaaatgtcgctatcctctaagctaacagtcaaggacctgagccttgcgggcaaacgcgtgttcatccgcgtggacttcaacgtgccgctagacgggaagacgatcacgtcgaaccagcgcattgtggccgcgctacccacgatcaagtacgtgttggagcagggccccaaggcggtggtgttggcgtcgcacctagggcggcccaacggcgagcgcaacgagaagtactcgttggcgccggtggccgcggagctggagaagctgctgggccagaaggtgaacttcctggacgactgcgtgggcgagcacgtgaccgctgcggtcaacggcgcggctgcggggtccgtgttcctgcttgagaacctgcggttccacatcgaggaggagggctcgcgcaaggtggacggcgagaaggtcaaggcgtctgcggaggacgtgcagaagttccgccagggcctcatgtcgctcgcggacgtctacgtcaacgacgccttcggcaccgcgcacagagcgcactcctcgatggtcggcttcgagctgcctgagagggccggcggcttcttgttgtcgcgcgagctggagtacttcagcaaggcgttggagaaccctacgagacccttcttggcgatcctgggcggtgccaaggttgccgacaagatccagttgatcgacaacttgttggacaaggtcgactcgattgtgatcggcggtggcatggccttcaccttcaagaaggtgcttgagaacatggagatcggtaactccatctacgacaaggcgggtgccgagatcgttcctaagctagcggagaaggccaagaagaacggcgtcaagatcgtgttgcctgtggacttcgtgatcggcgacgacttctcccaggacgccaacaccaagattgtctctgcctccgagggcattccatccggctgggagggtctagactgtggtcctgagtccagaaagctgttcagcgagaccatcgcgagcgcgaagaccatcgtgtggaacggcccaccaggtgtctttgagattccaaagttctccgagggtacccaggccatgcttgcggccgctgtcaaggcctccgaggctggcagcaccgtcatcatcggcggtggtgacaccgctaccgtggccaagaagtacggtgtggtcgagaagatctcccatgtgtccaccggtggtggtgcctccctagagctattggagggtaaggacctaccaggtgtcaccttcttgtccagcaagcagtagatgcgtccgcgcgcctgccggcttgaatagataataaactatatacgcaacgtaatggaaagttgagaaactactatatgcgcctgttgcgctgctcgtccgtgggctccagccgcgggaacccgagcagctccatgatgcagtactcgtccgggctctcgacgcacgcgccgctcgacttgtcaaacaggccgtgctgattcagcg

SEQ ID NO: 39 - protein, 448 aa, Ashbya gossypii
  misc_feature: Oligo 136
MGRSWGLLWMACLLYGVFTECRRSSQHAHTGKMSLSSKLTVKDLSLAGKRVFIRVDFNVPLDGKTITSNQRIVAALPTIKYVLEQGPKAVVLASHLGRPNGERNEKYSLAPVAAELEKLLGQKVNFLDDCVGEHVTAAVNGAAAGSVFLLENLRFHIEEEGSRKVDGEKVKASAEDVQKFRQGLMSLADVYVNDAFGTAHRAHSSMVGFELPERAGGFLLSRELEYFSKALENPTRPFLAILGGAKVADKIQLIDNLLDKVDSIVIGGGMAFTFKKVLENMEIGNSIYDKAGAEIVPKLAEKAKKNGVKIVLPVDFVIGDDFSQDANTKIVSASEGIPSGWEGLDCGPESRKLFSETIASAKTIVWNGPPGVFEIPKFSEGTQAMLAAAVKASEAGSTVIIGGGDTATVAKKYGVVEKISHVSTGGGASLELLEGKDLPGVTFLSSKQ

SEQ ID NO: 40 - DNA, 1006 bp, Ashbya gossypii
  misc_feature (361)..(361): n = unknown nucleotide
  misc_feature (388)..(388): n = unknown nucleotide
  misc_feature: Oligo 157
gatcgcgccgcaccacctcctcccacggcggccgcttctccggcgcaacaggaggcttgtggcggtcgtacaggcacctggtagtactgcaccttcacacgcgtgcctgtcgatccgcttgcgctgccaggtcatagaacttgggcaaattgaggtcccggtagtcccctccgttgtcgataaattggcgaagccggtcttcgtagatgccccttgacgggcttgaactgcgggtcccagttgaccgatatcgctgctcatagcgctgcttccttcctgccttggtccgtagtctcgcctgccgctcgcaccttttaatatccttacccgcgcggttttcgtaatgcagttttcscttgcngtycgsccgcagtcacccgaaaccctncccggaaaaacgcttttccccgcagggcgcgccatgaaacctgctgctgccggcctgttgtctgcctccggggcacgttgattattcagctatactgattatatatactctagttgtccaaaatgtgcatctcctggtccttctcgatgtgctggatcaagtcgtcgtgctgcgacttaatctgctccaaggccgtgacctcgggcaacttcacactgaggccgttgatgagcgagtgctcgtgcaggatactcccgccgatctgctcgatggactctctgaccttggcgaggtcggcccccttcttcaacgtgatgatataagatgattcagacatgcttggtgctgggactgatggtagtcatctacccgcgttcgcgtgcccttttatatacggccgagcccggcaaggggcccctaggcccccgtttcgcttgcgcaggccactgttgctggctacttacttaggcttactgtacytagattgctctgggctgtgcggtcagaggtgctgtaatcaggattgtcgagcgcacgccccagctcacgtgacagaacggccatcaggcgggaaccgtcgcgtagcgagacccactcgccgtcaatcagagccactag

SEQ ID NO: 41 - protein, 67 aa, Ashbya gossypii
  misc_feature: Oligo 157
SYIITLKKGADLAKVRESIEQIGGSILHEHSLINGLSVKLPEVTALEQIKSQHDDLIQHIEKDQEMH

SEQ ID NO: 42 - DNA, 1658 bp, Ashbya gossypii
  CDS (809)..(1033)
  misc_feature: Oligo 157
gttatgatgagccccgccagccccgcgggaaatagagcagaatgtacatgccgtgttttcgtccggacgtcctttcgtgcagttgtgtgcgactttttcagactttccgcggagccggcagcgcaacctcttcccccactatcgcaatgatccgccataccgcccccgttgcgcgccgtatcctgctcagtaggccttctgcactcctggcgcgccccgccgtacgcatcgccgcgaggccggccgcgtggcgcgccatgtcatccttctccggcggcatgcgcgctcccccggagctggcccagctcacgccgcaggcctaccacaagcaggccgacgagttcctgaacgacctgctggaccgtctcgaagcgctgggcgacgagcgccccgacatcatcgcagactccgagtactcccagggtgtgctcacgctgtccgtacctgctctgggcacctatgtcattaacaaacagcccccaaacaagcagatatggctctcgtcgccagtatccggccccaaccgctagtggctctgattgacggcgagtgggtctcgctacgcgacggttcccgcctgatggccgttctgtcacgtgagctggggcgtgcgctcgacaatcctgattacagcacctctgaccgcacagcccagagcaatctargtacagtagcacctaagtaagtagccagcaacagtggcctgcgcaagcgaaacgggggcctaggggccccttgccgggctcggccgtatataaaagggcacgcgaacgcgggtagatgactaccatcagtcccagcaccaagcatgtctgaatcatcttatatcatcacgttgaagaagggggccgacctcgccaaggtcagagagtccatcgagcagatcggcgggagtatcctgcacgagcactcgctcatcaacggcctcagtgtgaagttgcccgaggtcacggccttggagcagattaagtcgcagcacgacgacttgatccagcacatcgagaaggaccaggagatgcacattttggacaactagagtatatataatcagtatagctgaataatcaacgtgccccggaggcagacaacaggccggcagcagcaggtttcatggcgcgccctgcggggaagcgttttccgggaggtttcggtgactgcggccgacgcagcgaaactgcatacgaaaccgcgcggtaggatataaaggtgcgagcggcaggcgagactacggaccaaggcaggaaggagcagcgctatgagcagcgatatcgtcaactgggacccgcagttcaagcccgtcaagggcatctacgaagaccggcttcgccaatttatcgacaacggaggggactaccgggacctcaatttgcccaagttctatgaccggcagcgcaagcggatcgacagcacgcgtgtgaaggtgcagtactaccaggtgcctgtacgaccgccacaagcctcctgttgcgccggagaagcggccgccgtgggaggaggtggtgcggcgcgatcgtgctggagaaatcgaatggaagatgtgtacctcgaccccgtcgggccgagctggtccactacgaggttctaggtgagttggacatcccggaatcgtggagcagctctggggaacagataatattctagtgggactgcgagaacaa

SEQ ID NO: 43 - protein, 75 aa, Ashbya gossypii
  misc_feature: Oligo 157
MSESSYIITLKKGADLAKVRESIEQIGGSILHEHSLINGLSVKLPEVTALEQIKSQHDDLIQHIEKDQEMHILDN

SEQ ID NO: 44 - DNA, 1599 bp, Ashbya gossypii
  misc_feature (331)..(331): n = unknown nucleotide
  misc_feature (343)..(343): n = unknown nucleotide
  misc_feature: Oligo 108
gatcgatatacaggttttggggcatttgcaactcctcttgcgggatatgtcaccgctgcggcatccgaacaggggccctgaattactggcggttgacccagatctgttcagccgcatgttcttcacgaagccgtctcaggattcgctgttcctgaagagcaagggaccattgccgtcgcggattcttccccatttgtaccttggttcactcgagcacgcgcatgacccaaacctgctccgcagattgggtatcagaaatattgtgtcagtcggggagactttatcatggctacaatcccctcactcccgggaggctgctaaaacagaaganagcaaacaaganaccatcacggacgcgtatgtgaccgcacctgctgaaaccgccgcggcaacgtccacgaccgcatccactggtagtcttaatttgttgaagcgaaacagatcacccagcgcgcctgaaattaaaccgttaaaatcctcatcctgtaaaactacagtcttctcgaaggatggattcgaaatatgccacacaagcaacttgggagacaatggtgcagatccgatgctcactcagctggaccaaatgctctctttcatcaatcggtgttatgaggccggcgaaaaggtactggttcactgcatggtcggcgtttctcgcagtgcgacagtatgcatagccgaatgcatgagacgtctaaactgcgcagtgatccaggcctacctgtttgtccgtgtgcgccgcttaaacattataatccagcccaatctcatgttcatctacgagctcctcaagtggcaggagctcaagcaccctgacgttctcaagattgactggcatatcatgtgtagggccatcaacgaactcaacagtaattatataaaggacatgatttgacctaccagttgagcttggttgcccactctcacattttgtactatttagaggattttacgataccataaactactctagtgtaacatatttcttcaacgagtccggaatctcatcgtagagcttcttttcctgcaaccacttcttcgagaatacgcggtcggcatacttgtgtccagtgtcacagaggatcgtgacgacattgcttcccggtggtagttccctggccacacgcacagcgccagcgacgttcagccccgaagtgccaccgaggaatagaccttcctcgtccagaagcctgtaggtcatgttcagggactctgggtccggaacaaacacggcgtcgtcaatgatatcaacgctggcttcgaggttctttgtaatccgcccttggcccacgccctcagtgaacgagctgccctccctctccaagttgccggtcttgacgaatgaatagacgaccgagcctggcgggtccgtgaccaccagtttgcatttgcgctccgtggcctcggccaggtaccgcgcgatcccggagaacgttcctcctggtacctgtggagcaaggtgaatgcatccacctcgagcccagcgttgcgcatttgctgcaagatctcgggaccggtcgtcgcatagtgcgccttccaattggcctcgttgtcaaactggttggtccagacagcattgtccaatgccgcggcgtgatc

SEQ ID NO: 45 - protein, 46 aa, Ashbya gossypii
  misc_feature: Oligo 108
HAAALDNAVWTNQFDNEANWKAHYATTGPEILQQMRNAGLEVDAFT

SEQ ID NO: 46 - protein, 157 aa, Ashbya gossypii
  misc_feature: Oligo 108
GGTFSGIARYLAEATERKCKLVVTDPPGSVVYSFVKTGNLEREGSSFTEGVGQGRITKNLEASVDIIDDAVFVPDPESLNMTYRLLDEEGLFLGGTSGLNVAGAVRVARELPPGSNVVTILCDTGHKYADRVFSKKWLQEKKLYDEIPDSLKKYVTL

SEQ ID NO: 47 - DNA, 2808 bp, Ashbya gossypii
  CDS (450)..(1517)
  misc_feature: Oligo 108
ccgctgacggtgcgcaacctggtcaagtggcgcacggcaaggagctggagcagttcctggcggacgtggcggacgtgcggcggtgttgctcgagcatcagggcgccgcggactcgccggaagaggcggagcgctttgtgggcggcctgcgcagcgcccttgaggcgccggcggcggcgcctgcgccgccagcgcgcaagagatacgagttcgctccccggcccgcgcgcaactgaaccgcgcacctgtatataccgcgacatagtgaataaagaatgagagtcccacagtgcgcctgcgcgcggagcgcgcggcatggtcgccgaccgctgttgtgccgccgcacgtgattagtgcactatggctgtggatctgtgctgatgtcgggtcaccgtccagtcgctgggtatatcagtacagcagtaggcagcccggccggaacacgttacgatgcacgcccagctcattaggaagatgtcagcgcccacactggccgccggaggcttcccggatgccattggcaagacgccgctgatcaagctgcagaagttgtcgcaggaactgggtcggaacatctacgggaaggcggagtttatgaacccgggcggatccgtcaaggaccgtgctgcgctttacctggtggaagaagcggaacgggcggggctgatcgacccttcgcggccggggaccatagtagagggctctgccggcaacacggcgatcgggctggcacacatagcacgctccagggggtacaatgctgtgttctacatgccggacacgcagtcgcgggaaaaggtcaacttgctgaggtacctggggaccgaggtgcacccggtacccgtgtgccccagcacagaccctctgaacttcaacaatcgcgctcgggatcacgccgcggcattggacaatgctgtctggaccaaccagtttgacaacgaggccaattggaaggcgcactatgcgacgaccggtcccgagatcttgcagcaaatgcgcaacgctgggctcgaggtggatgcattcacttgctccacaggtaccggaggaacgttctccgggatcgcgcggtacctggccgaggccacggagcgcaaatgcaaactggtggtcacggacccgccaggctcggtcgtctattcattcgtcaagaccggcaacttggagagggagggcagctcgttcactgagggcgtgggccaagggcggattacaaagaacctcgaagccagcgttgatatcattgacgacgccgtgtttgttccggacccagagtccctgaacatgacctacaggcttctggacgaggaaggtctattcctcggtggcacttcggggctgaacgtcgctggcgctgtgcgtgtggccagggaactaccaccgggaagcaatgtcgtcacgatcctctgtgacactggacacaagtatgccgaccgcgtattctcgaagaagtggttgcaggaaaagaagctctacgatgagattccggactcgttgaagaaatatgttacactagagtagtttatggtatcgtaaaatcctctaaatagtacaaaatgtgagagtgggcaaccaagctcaactggtaggtcaaatcatgtcctttatataattactgttgagttcgttgatggccctacacatgatatgccagtcaatcttgagaacgtcagggtgcttgagctcctgccacttgaggagctcgtagatgaacatgagattgggctggattataatgtttaagcggcgcacacggacaaacaggtaggcctggatcactgcgcagtttagacgtctcatgcattcggctatgcatactgtcgcactgcgagaaacgccgaccatgcagtgaaccagtaccttttcgccggcctcataacaccgattgatgaaagagagcatttggtccagctgagtgagcatcggatctgcaccattgtctcccaagttgcttgtgtggcatatttcgaatccatccttcgagaagactgtagttttacaggatgaggattttaacggtttaatttcaggcgcgctgggtgatctgtttcgcttcaacaaattaagactaccagtggatgcggtcgtggacgttgccgcggcggtttcagcaggtgcggtcacatacgcgtccgtgatggttcttgtttgcttcttctgttttagcagcctcccgggagtgaggggattgtagccatgataaagtctccccgactgacacaatatttctgatacccaatctgcggagcaggtttgggtcatgcgcgtgctcgagtgaaccaaggtacaaatggggaagaatccgcgacggcaatggtcccttgctcttcaggaacagcgaatcctgagacggcttcgtgaagaacatgcggctgaacagatctgggtcaaccgccagtaattcagggcccctgttcggatgccgcagcggtgacatatcccgcaagaggagttgcaaatgccccaaaacctgtatatcgatcggaaataggaagtatggacgctcgacctccgtatgcaaccgcaacagaacctcatccagaggttgatcccaggcaaatatcagataagccactagcaaaaaggaagtttctgtataaccatcggaacagtataatagggaaagcttcttgtgtaccgcgctgaatcggtagatcaggtgacagatattaacacacgaagtgatggagtccagagtgagggtccccaggccaattgagcccgagctcggaaacgcaatgtgtatcggagcctccatctgggtcaagtgatgcctgatgtcgtcgtggtgtgggaagggcgtgtgctc

SEQ ID NO: 48 - protein, 356 aa, Ashbya gossypii
  misc_feature: Oligo 108
MHAQLIRKMSAPTLAAGGFPDAIGKTPLIKLQKLSQELGRNIYGKAEFMNPGGSVKDRAALYLVEEAERAGLIDPSRPGTIVEGSAGNTAIGLAHIARSRGYNAVFYMPDTQSREKVNLLRYLGTEVHPVPVCPSTDPLNFNNRARDHAAALDNAVWTNQFDNEANWKAHYATTGPEILQQMRNAGLEVDAFTCSTGTGGTFSGIARYLAEATERKCKLVVTDPPGSVVYSFVKTGNLEREGSSFTEGVGQGRITKNLEASVDIIDDAVFVPDPESLNMTYRLLDEEGLFLGGTSGLNVAGAVRVARELPPGSNVVTILCDTGHKYADRVFSKKWLQEKKLYDEIPDSLKKYVTLE

(19) United States
(12) Patent Application Publication
(10) Pub. No.: US 2007/0004015 A1
(43) Pub. Date: Jan. 4, 2007

(54) Novel metabolism-associated gene products from ashbya gossypii

(76) Inventors: Marvin Karos, Neustadt (DE); Henning Althofer, Wachenheim (DE); Burkhard Kroger, Limburgerhof (DE); Jose L. Revuelta Doval, Salamanca (ES)

Correspondence Address: MORRISON & FOERSTER LLP, 1650 TYSONS BOULEVARD, SUITE 300, MCLEAN, VA, 22102, US

(21) Appl. No.: 10/487,476

(22) PCT Filed: Aug. 23, 2002
(86) PCT No.: PCT/EP02/09454

§ 371 Date: Feb. 23, 2004

(30) Foreign Application Priority Data
Aug. 23, 2001 (DE) 10141267.3
Aug. 23, 2001 (DE) 10141268.1

(51) Int. Cl. C12P 17/00; C07H 21/04; C12P 21/06; C12N 9/10; C12N 15/74; C12N 1/21; C07K 16/40

(52) U.S. Cl. 435/117; 435/193; 435/69.1; 435/252.3; 435/471; 536/23.2; 530/388.26

(57) ABSTRACT

The present invention relates to novel polynucleotides from Ashbya gossypii; to oligonucleotides hybridizing therewith; to expression cassettes and vectors which comprise these polynudeotides; to microorganisms transformed therewith; to polypeptides encoded by these polynucleotides; and to the use of the novel polypeptides and polynucleotides as targets for modulating the metabolic reactions and, in particular, improving vitamin B2 production in microorganisms of the genus Ashbya.

Novel metabolism-associated gene products from Ashbya gossypii.

The present invention relates to novel polynucleotides from Ashbya gossypii; to oligonucleotides hybridizing therewith; to expression cassettes and vectors which comprise these polynucleotides; to microorganisms transformed therewith; to polypeptides encoded by these polynucleotides; and to the use of the novel polypeptides and polynucleotides as targets for modulating metabolism and, in particular, improving vitamin B2 production in microorganisms of the genus Ashbya.

Vitamin B2 (riboflavin, lactoflavin) is an alkali- and light-sensitive vitamin which shows a yellowish green fluorescence in solution. Vitamin B2 deficiency may lead to ectodermal damage, in particular cataract, keratitis, corneal vascularization, or to autonomic and urogenital disorders. Vitamin B2 is a precursor for the molecules FAD and FMN which, besides NAD⁺ and NADP⁺, are important in biology for hydrogen transfer. They are formed from vitamin B2 by phosphorylation (FMN) and subsequent adenylation (FAD).

Vitamin B2 is synthesized in plants, yeasts and many microorganisms from GTP and ribulose 5-phosphate. The reaction pathway starts with opening of the imidazole ring of GTP and elimination of a phosphate residue. Deamination, reduction and elimination of the remaining phosphate result in 5-amino-6-ribitylamino-2,4-pyrimidinone. Reaction of this compound with 3,4-dihydroxy-2-butanone 4-phosphate leads to the bicyclic molecule 6,7-dimethyl-8-ribityllumazine. This compound is converted into the tricyclic compound riboflavin by dismutation, in which a 4-carbon unit is transferred.

Vitamin B2 occurs in many vegetables and in meat, and to a lesser extent in cereal products. The daily vitamin B2 requirement of an adult is about 1.4 to 2 mg. The main breakdown product of the coenzymes FMN and FAD in humans is in turn riboflavin, which is excreted as such.

Vitamin B2 is thus an important dietary substance for humans and animals. Efforts are therefore being made to make vitamin B2 available on the industrial scale. It has therefore been proposed to synthesize vitamin B2 by a microbiological route. Microorganisms which can be used for this purpose are, for example, Bacillus subtilis, the ascomycetes Eremothecium ashbyii, Ashbya gossypii, and the yeasts Candida flareri and Saccharomyces cerevisiae. The nutrient media used for this purpose comprise molasses or vegetable oils as carbon source, inorganic salts, amino acids, animal or vegetable peptones and proteins, and vitamin additions. In sterile aerobic submerged processes, yields of more than 10 g of vitamin B2 are obtained per liter of culture broth within a few days. The requirements are good aeration of the culture, careful agitation and setting of temperatures below about 30° C. Removal of the biomass, evaporation and drying of the concentrate result in a product enriched in vitamin B2.

Microbiological production of vitamin B2 is described, for example, in WO-A-92/01060, EP-A-0 405 370 and EP-A-0 531 708.

A survey of the importance, occurrence, production, biosynthesis and use of vitamin B2 is to be found, for example, in Ullmann's Encyclopaedia of Industrial Chemistry, volume A27, pages 521 et seq.

The overall process by which living systems acquire and utilize the free enthalpy which is required to carry out their various functions is referred to as metabolism. The pathways of metabolism consist of sequences of enzymatic reactions which provide specific products. The metabolic reaction pathways are frequently divided into two categories. Firstly the pathways involved in degradation (catabolism) and secondly the pathways involved in biosynthesis (anabolism). In the catabolic pathways, complex metabolites are broken down exergonically into simpler products. The free enthalpy released in these processes is stored through synthesis of high-energy compounds (ATP, NADPH) which can be employed universally in the cell. ATP and NADPH are the principal sources of free enthalpy for many biosynthetic reactions in the anabolic pathways. For example, NADPH and ATP are required in numerous synthetic steps to produce fine chemicals, such as in the case of riboflavin synthesis.

In the catabolic pathways, a large number of different substances (carbohydrates, lipids and proteins) is converted into the same intermediate products. The reverse process takes place during biosynthesis. Relatively few metabolites serve as starting substances for a large number of different biosynthetic products. It is possible through controlled intervention or blocking of pathways of degradation of fine chemicals which have already been synthesized to additionally increase production thereof.

Some products and byproducts of these naturally occurring metabolic processes have found industrial applications in a wide area, such as, for example, the human and animal food industries, and the cosmetic and pharmaceutical industries. These molecules, given the comprehensive name “fine chemicals”, include organic acids, proteinogenic and non-proteinogenic amino acids, nucleotides and nucleosides, lipids and fatty acids, diols, carbohydrates, aromatic compounds, vitamins and cofactors, and enzymes. These substances are mostly produced in large-volume fermenters in which the molecules which are required in each case are excreted into the medium in high concentrations. A particularly useful organism for this process is the filamentous ascomycete Ashbya gossypii.

An alteration in proteins involved in these metabolic pathways, in terms of the amount and/or the activity, may have a direct effect on the production or the efficiency of production of a desired fine chemical. For example, a reaction which proceeds in direct competition to an intermediate product which appears for the desired fine chemical can be eliminated, or a metabolic pathway responsible for producing this specific intermediate product can be optimized.

The use of genes of the metabolism for generating microorganisms, preferably of the genous Ashbya, in particular of Ashbya gossypii strains, with modulated metabolism has not yet been described.

It is an object of the present invention to provide novel targets for influencing metabolic processes in microorganisms of the genus Ashbya, in particular in Ashbya gossypii. The object in particular is to optimise specifically particular metabolic pathways in such microorganisms. A further object is to improve the vitamin B2 production by such microorganisms.

We have found that this object is achieved by providing encoding nucleic acid sequences which are up- or downregulated in Ashbya gossypii during vitamin B2 production (based on results found with the aid of the MPSS analytical method described in detail in the experimental part), in particular:

a) a, preferably downregulated, nucleic acid sequence which codes for a protein having the function of a C1-tetrahydrofolate synthase.

In a preferred embodiment of this aspect of the invention there has been isolation of a DNA clone which codes for a characteristic part-sequence of the nucleic acid sequence of the invention and which bears the internal name “Oligo 72”.

In a further preferred embodiment there has been isolation according to the invention of a DNA clone which codes for the complete sequence of the nucleic acid of the invention and which bears the internal name “Oligo 72v”.

A first aspect of the present invention relates to a polynucleotide comprising a nucleic acid sequence as shown in SEQ ID NO: 1. A further aspect of the invention relates to a polynucleotide comprising a nucleic acid sequence as shown in SEQ ID NO: 4 or a fragment thereof. The polynucleotides can be isolated preferably from a microorganism of the genus Ashbya, in particular A. gossypii. The invention additionally relates to the polynucleotides complementary thereto; and to the sequences derived from these polynucleotides through the degeneracy of the genetic code.

The inserts of “Oligo 72” and “Oligo 72v” have significant homologies with the MIPS tag “Ade3” from S. cerevisiae. The inserts have a nucleic acid sequence as shown in SEQ ID NO: 1 or SEQ ID NO: 4. The amino acid sequence or amino acid part-sequence derived from the coding strand as shown in SEQ ID NO: 1 or 4 has significant sequence homology with a C1 -tetrahydrofolate synthase from S. cerevisiae.

b) a, preferably upregulated, nucleic acid sequence which codes for a protein having the function of an argininosuccinate synthase.

In a preferred embodiment of this aspect of the invention there has been isolation of a DNA clone which codes for a characteristic part-sequence of the nucleic acid sequence of the invention and which bears the internal name “Oligo 81”.

In a further preferred embodiment there has been isolation according to the invention of a DNA clone which codes for the complete sequence of the nucleic acid of the invention and which bears the internal name =37 Oligo 81v”.

A first aspect of the present invention relates to a polynucleotide comprising a nucleic acid sequence as shown in SEQ ID NO: 6. A further aspect of the invention relates to a polynucleotide comprising a nucleic acid sequence as shown in SEQ ID NO: 9 or a fragment thereof. The polynucleotides can be isolated preferably from a microorganism of the genus Ashbya, in particular A. gossypii. The invention additionally relates to the polynucleotides complementary thereto; and to the sequences derived from these polynucleotides through the degeneracy of the genetic code.

The inserts of “Oligo 81” and “Oligo 81v” have significant homologies with the MIPS tag “Arg1” from S. cerevisiae. The inserts have a nucleic acid sequence as shown in SEQ ID NO: 6 or SEQ ID NO: 9. The amino acid sequence or amino acid part-sequence derived from the corresponding complementary strand to SEQ ID NO: 6 or from the coding strand as shown in SEQ ID NO: 9 has significant sequence homology with an argininosuccinate synthase from S. cerevisiae.

c) a, preferably upregulated, nucleic acid sequence which codes for a protein having the function of a phosphoribosylaminoimidazole-succinocarboxamide synthase.

In a preferred embodiment of this aspect of the invention there has been isolation of a DNA clone which codes for a characteristic part-sequence of the nucleic acid sequence of the invention and which bears the internal name “Oligo 86”.

In a further preferred embodiment there has been isolation according to the invention of a DNA clone which codes for the complete sequence of the nucleic acid of the invention and which bears the internal name “Oligo 86v”.

A first aspect of the present invention relates to a polynucleotide comprising a nucleic acid sequence as shown in SEQ ID NO: 12. A further aspect of the invention relates to a polynucleotide comprising a nucleic acid sequence as shown in SEQ ID NO: 14 or a fragment thereof. The polynucleotides can be isolated preferably from a microorganism of the genus Ashbya, in particular A. gossypii. The invention additionally relates to the polynucleotides complementary thereto; and to the sequences derived from these polynucleotides through the degeneracy of the genetic code.

The inserts of “Oligo 86” and “Oligo 86v” have significant homologies with the MIPS tag =37 Ade1” from S. cerevisiae. The inserts have a nucleic acid sequence as shown in SEQ ID NO: 12 or SEQ ID NO: 14. The amino acid sequence or amino acid part-sequence derived from the corresponding complementary strand to SEQ ID NO: 12 or from the coding strand as shown in SEQ ID NO: 14 has significant sequence homology with a phosphoribosylaminoimidazole-succinocarboxamide synthase from S. cerevisiae.

d) a, preferably downregulated, nucleic acid sequence which codes for a protein having the function of a fumarate reductase.

In a preferred embodiment of this aspect of the invention there has been isolation of a DNA clone which codes for a characteristic part-sequence of the nucleic acid sequence of the invention and which bears the internal name “Oligo 162”.

In a further preferred embodiment there has been isolation according to the invention of a DNA clone which codes for the complete sequence of the nucleic acid sequence of the invention and which bears the internal name “Oligo 162v”.

A first aspect of the present invention relates to a polynucleotide comprising a nucleic acid sequence as shown in SEQ ID NO: 16. A further aspect of the invention relates to a polynucleotide comprising a nucleic acid sequence as shown in SEQ ID NO: 18 or a fragment thereof. The polynucleotides can be isolated preferably from a microorganism of the genus Ashbya, in particular A. gossypii. The invention additionally relates to the polynucleotides complementary thereto; and to the sequences derived from these polynucleotides through the degeneracy of the genetic code.

The inserts of “Oligo 162” and “Oligo 162v” have significant homologies with the MIPS tag “FRDS1 (2)” from S. cerevisiae. The inserts have a nucleic acid sequence as shown in SEQ ID NO: 16 or SEQ ID NO: 18. The amino acid sequence or amino acid part-sequence derived from the coding strand has significant sequence homology with a fumarate reductase from S. cerevisiae.

e) a, preferably upregulated, nucleic acid sequence which codes for a protein having the function of a phosphoenolpyruvate carboxykinase.

In a preferred embodiment of this aspect of the invention there has been isolation of a DNA clone which codes for a characteristic part-sequence of the nucleic acid sequence of the invention and which bears the internal name “Oligo 178”.

In a further preferred embodiment there has been isolation according to the invention of a DNA clone which codes for the complete sequence of the nucleic acid of the invention and which bears the internal name “Oligo 178v”.

A first aspect of the present invention relates to a polynucleotide comprising a nucleic acid sequence as shown in SEQ ID NO: 20. A further aspect of the invention relates to a polynucleotide comprising a nucleic acid sequence as shown in SEQ ID NO: 22 or a fragment thereof. The polynucleotides can be isolated preferably from a microorganism of the genus Ashbya, in particular A. gossypii. The invention additionally relates to the polynucleotides complementary thereto; and to the sequences derived from these polynucleotides through the degeneracy of the genetic code.

The inserts of “Oligo 178” and “Oligo 178v” have significant homologies with the MIPS tag “PCK1” from S. cerevisiae. The inserts have a nucleic acid sequence as shown in SEQ ID NO: 20 or SEQ ID NO: 22. The amino acid sequences derived from the corresponding complementary strand to SEQ ID NO: 20 or from the coding strand as shown in SEQ ID NO: 22 have significant sequence homology with a phosphoenolpyruvate carboxykinase from S. cerevisiae.

f) a, preferably upregulated, nucleic acid sequence which codes for a protein having the function of a uroporphyrinogen decarboxylase.

In a preferred embodiment of this aspect of the invention there has been isolation of a DNA clone which codes for a characteristic part-sequence of the nucleic acid sequence of the invention and which bears the internal name “Oligo 64”.

In a further preferred embodiment there has been isolation according to the invention of a DNA clone which codes for the complete sequence of the nucleic acid of the invention and which bears the internal name “Oligo 64v”.

A first aspect of the present invention relates to a polynucleotide comprising a nucleic acid sequence as shown in SEQ ID NO: 24. A further aspect of the invention relates to a polynucleotide comprising a nucleic acid sequence as shown in SEQ ID NO: 26 or a fragment thereof. The polynucleotides can be isolated preferably from a microorganism of the genus Ashbya, in particular A. gossypii. The invention additionally relates to the polynucleotides complementary thereto; and to the sequences derived from these polynucleotides through the degeneracy of the genetic code.

The inserts of “Oligo 64” and “Oligo 64v” have significant homologies with the MIPS tag “Hem12” from S. cerevisiae. The inserts have a nucleic acid sequence as shown in SEQ ID NO: 24 or SEQ ID NO: 26. The amino acid sequence or amino acid part-sequence derived from the coding strand has significant sequence homology with a uroporphyrinogen decarboxylase from S. cerevisiae.

g) a, preferably upregulated, nucleic acid sequence which codes for a protein having the function of a siroheme synthase.

In a preferred embodiment of this aspect of the invention there has been isolation of a DNA clone which codes for a characteristic part-sequence of the nucleic acid sequence of the invention and which bears the internal name “Oligo 125”.

In a further preferred embodiment there has been isolation according to the invention of a DNA clone which codes for the complete sequence of the nucleic acid of the invention and which bears the internal name “Oligo 125v”.

A first aspect of the present invention relates to a polynucleotide comprising a nucleic acid sequence as shown in SEQ ID NO: 28. A further aspect of the invention relates to a polynucleotide comprising a nucleic acid sequence as shown in SEQ ID NO: 30 or a fragment thereof. The polynucleotides can be isolated preferably from a microorganism of the genus Ashbya, in particular A. gossypii. The invention additionally relates to the polynucleotides complementary thereto; and to the sequences derived from these polynucleotides through the degeneracy of the genetic code.

The inserts of “Oligo 125” and “Oligo 125v” have significant homologies with the MIPS tag “Met1” from S. cerevisiae. The inserts have a nucleic acid sequence as shown in SEQ ID NO: 28 or SEQ ID NO: 30. The amino acid sequence or amino acid part-sequence derived from the complementary strand to SEQ ID NO: 28 or from the coding strand shown in SEQ ID NO: 30 has significant sequence homology with a siroheme synthase from S. cerevisiae.

h) a, preferably upregulated, nucleic acid sequence which codes for a protein having the function of a uroporphyrinogen III synthase.

In a preferred embodiment of this aspect of the invention there has been isolation of a DNA clone which codes for a characteristic part-sequence of the nucleic acid sequence of the invention and which bears the internal name “Oligo 107”.

In a further preferred embodiment there has been isolation according to the invention of a DNA clone which codes for the complete sequence of the nucleic acid of the invention and which bears the internal name =37 Oligo 107v”.

A first aspect of the present invention relates to a polynucleotide comprising a nucleic acid sequence as shown in SEQ ID NO: 32. A further aspect of the invention relates to a polynucleotide comprising a nucleic acid sequence as shown in SEQ ID NO: 34 or a fragment thereof. The polynucleotides can be isolated preferably from a microorganism of the genus Ashbya, in particular A. gossypii. The invention additionally relates to the polynucleotides complementary thereto; and to the sequences derived from these polynucleotides through the degeneracy of the genetic code.

The inserts of “Oligo 107” and “Oligo 107v” have significant homologies with the MIPS tag “Hem4” from S. cerevisiae. The inserts have a nucleic acid sequence as shown in SEQ ID NO: 32 or SEQ ID NO: 34. The amino acid sequence or amino acid part-sequence derived from the coding strand has significant sequence homology with a uroporphyrinogen III synthase from S. cerevisiae.

i) a, preferably downregulated, nucleic acid sequence which codes for a protein having the function of an argininosuccinate synthase.

In a preferred embodiment of this aspect of the invention there has been isolation of a DNA clone which codes for a characteristic part-sequence of the nucleic acid sequence of the invention and which bears the internal name “Oligo 136”.

In a further preferred embodiment there has been isolation according to the invention of a DNA clone which codes for the complete sequence of the nucleic acid of the invention and which bears the internal name “Oligo 136v”.

A first aspect of the present invention relates to a polynucleotide comprising a nucleic acid sequence as shown in SEQ ID NO: 36. A further aspect of the invention relates to a polynucleotide comprising a nucleic acid sequence as shown in SEQ ID NO: 38 or a fragment thereof. The polynucleotides can be isolated preferably from a microorganism of the genus Ashbya, in particular A. gossypii. The invention additionally relates to the polynucleotides complementary thereto; and to the sequences derived from these polynucleotides through the degeneracy of the genetic code.

The inserts of “Oligo 136” and “Oligo 136v” have significant homologies with the MIPS tag “Pgk1” from S. cerevisiae. The inserts have a nucleic acid sequence as shown in SEQ ID NO: 36 or SEQ ID NO: 38. The amino acid sequences derived in each case from the coding strand have significant sequence homology with a phosphoglycerate kinase from S. cerevisiae.

k) a, preferably downregulated, nucleic acid sequence which codes for a protein having the function of a proteinase B inhibitor 2.

In a preferred embodiment of this aspect of the invention there has been isolation of a DNA clone which codes for a characteristic part-sequence of the nucleic acid sequence of the invention and which bears the internal name “Oligo 157”.

In a further preferred embodiment there has been isolation according to the invention of a DNA clone which codes for the complete sequence of the nucleic acid of the invention and which bears the internal name “Oligo 157v”.

A first aspect of the present invention relates to a polynucleotide comprising a nucleic acid sequence as shown in SEQ ID NO: 40. A further aspect of the invention relates to a polynucleotide comprising a nucleic acid sequence as shown in SEQ ID NO: 42 or a fragment thereof. The polynucleotides can be isolated preferably from a microorganism of the genus Ashbya, in particular A. gossypii. The invention additionally relates to the polynucleotides complementary thereto; and to the sequences derived from these polynucleotides through the degeneracy of the genetic code.

The inserts of “Oligo 157” and “Oligo 157v” have significant homologies with the MIPS tag “PB12” from S. cerevisiae. The inserts have a nucleic acid sequence as shown in SEQ ID NO: 40 or SEQ ID NO: 42. The amino acid sequences derived from the corresponding complementary strand to SEQ ID NO: 40 or from the coding strand of SEQ ID NO: 42 have significant sequence homology with a proteinase B inhibitor 2 from S. cerevisiae.

I) a, preferably upregulated, nucleic acid sequence wich codes for a protein having the function of a cysteine synthase.

In a preferred embodiment of this aspect of the invention there has been isolation of a DNA clone which codes for a characteristic part-sequence of the nucleic acid sequence of the invention and which bears the internal name “Oligo 108”.

In a further preferred embodiment there has been isolation according to the invention of a DNA clone which codes for the complete sequence of the nucleic acid of the invention and which bears the internal name “Oligo 108v”.

A first aspect of the present invention relates to a polynucleotide comprising a nucleic acid sequence as shown in SEQ ID NO: 44. A further aspect of the invention relates to a polynucleotide comprising a nucleic acid sequence as shown in SEQ ID NO: 47 or a fragment thereof. The polynucleotides can be isolated preferably from a microorganism of the genus Ashbya, in particular A. gossypii. The invention additionally relates to the polynucleotides complementary thereto; and to the sequences derived from these polynucleotides through the degeneracy of the genetic code.

The inserts of “Oligo 108” and “Oligo 108v” have significant homologies with the MIPS tag “CYSK” from S. cerevisiae. The inserts have a nucleic acid sequence as shown in SEQ ID NO: 44 or SEQ ID NO: 47. The amino acid sequence or amino acid part-sequence derived from the corresponding complementary strand of SEQ ID NO: 44 or from the coding strand shown in SEQ ID NO: 47 has significant sequence homology with a cysteine synthase from A. nidulans.

A further aspect of the invention relates to oligonucleotides which hybridize with one of the above polynucleotides, in particular under stringent conditions.

The invention additionally relates to polynucleotides which hybridize with one of the oligonucleotides of the invention and code for a gene product from microorganisms of the genus Ashbya or a functional equivalent of this gene product.

The invention further relates to polypeptides or proteins which are encoded by the polynucleotides described above; and to peptide fragments thereof which have an amino acid sequence which comprises at least 10 consecutive amino acid residues as shown in SEQ ID NO: 2, 3, 5, 7, 8, 10, 11, 13, 15, 17, 19, 21, 23, 25, 27, 29, 31, 33, 35, 37, 39, 41, 43, 45, 46 or SEQ ID NO: 48; and to functional equivalents of the polypeptides or proteins of the invention.

In this connection, functional equivalents differ from the products specifically disclosed in the invention by their amino acid sequence through addition, insertion, substitution, deletion or inversion at a minimum of one, such as, for example, 1 to 30 or 1 to 20 or 1 to 10, sequence positions without the originally observed protein function, which can be deduced by sequence comparison with other proteins, being lost. It is thus possible for equivalents to have essentially identical, higher or lower activities compared with the native protein.

Further aspects of the invention relate to expression cassettes for the recombinant production of proteins of the invention, comprising one of the nucleic acid sequences defined above, operatively linked to at least one regulatory nucleic acid sequence; and to recombinant vectors comprising at least one such expression cassette of the invention.

Also provided according to the invention are prokaryotic or eukaryotic hosts which are transformed with at least one vector of the above type. A preferred embodiment provides prokaryotic or eukaryotic hosts in which the functional expression of at least one gene which codes for a polypeptide of the invention as defined above is modulated (e.g. inhibited or overexpressed); or in which the biological activity of a polypeptide as defined above is reduced or increased. Preferred hosts are selected from ascomycetes, in particular those of the genus Ashbya and preferably strains of A. gossypii.

Modulation of gene expression in the above sense includes both inhibition thereof, for example through blockade of a stage in expression (in particular transcription or translation) or a specific overexpression of a gene (for example through modification of regulatory sequences or increasing the copy number of the coding sequence).

A further aspect of the invention relates to the use of an expression cassette of the invention, of a vector of the invention or of a host of the invention for the microbiological production of vitamin B2 and/or precursors and/or derivatives thereof. A further aspect of the invention relates to the use of an expression cassette of the invention, of a vector of the invention or of a host of the invention for the recombinant production of a polypeptide of the invention as defined above.

Also provided according to the invention is a method for detecting or for validating an effector target for modulating the microbiological production of vitamin B2 and/or precursors and/or derivatives thereof. This entails treating a microorganism capable of the microbiological production of vitamin B2 and/or precursors and/or derivatives thereof with an effector which interacts with (such as, for example, non-covalently binds to) a target selected from a polypeptide of the invention as defined above or a nucleic acid sequence coding therefor, validating the influence of the effector on the amount of the microbiologically produced vitamin B2 and/or of the precursor and/or of a derivative thereof; and isolating the target where appropriate. The validation in this case takes place preferably by direct comparison with the microbiological vitamin B2 production in the absence of the effector under otherwise identical conditions.

A further aspect of the invention relates to a method for modulating (in relation to the amount and/or rate of) the microbiological production of vitamin B2 and/or precursors and/or derivatives thereof, where a microorganism capable of the microbiological production of vitamin B2 and/or precursors and/or derivatives thereof is treated with an effector which interacts with a target selected from a polypeptide of the invention as defined above or a nucleic acid sequence coding therefor.

Preferred examples of the abovementioned effectors which should be mentioned are:
a) antibodies or antigen-binding fragments thereof;
b) polypeptide ligands which are different from a) and which interact with a polypeptide of the invention;
c) low molecular weight effectors which modulate the biological activity of a polypeptide of the invention;
d) antisense nucleic acid sequences which interact with a nucleic acid sequence of the invention.

The invention likewise relates to abovementioned effectors having specificity for at least one of the targets, according to the invention, defined above.

A further aspect of the invention relates to a method for the microbiological production of vitamin B2 and/or precursors and/or derivatives thereof, where a host as defined above is cultivated under conditions favoring the production of vitamin B2 and/or precursors and/or derivatives thereof, and the desired product(s) is (are) isolated from the culture mixture. It is preferred in this connection that the host is treated with an effector as defined above before and/or during the cultivation. A preferred host is in this case selected from microorganisms of the genus Ashbya; in particular transformed as described above.

A final aspect of the invention relates to the use of a polynucleotide or polypeptide of the invention as target for modulating the production of vitamin B2 and/or precursors and/or derivatives thereof in a microorganism of the genus Ashbya.

### DESCRIPTION OF THE FIGURES

FIG. 1 shows an alignment between an amino acid part-sequence of the invention (corresponding to the strand of position 1 to 144 in SEQ ID NO: 1) (upper sequence) and a part-sequence of the MIPS tag Ade3 from S. cerevisiae (lower sequence). Identical sequence positions are indicated between the two sequences. Similar sequence positions are labeled with “+”.

FIG. 2 A shows a further alignment between an amino acid part-sequence of the invention (corresponding to the complementary strand to position 862 to 551 in SEQ ID NO: 6) (upper sequence) and a part-sequence of the MIPS tag Arg1 from S. cerevisiae (lower sequence). FIG. 2B shows an alignment between an amino acid part-sequence of the invention (corresponding to the complementary strand to position 912 to 859 in SEQ ID NO: 6) (upper sequence) and a part-sequence of the MIPS tag Arg1 from S. cerevisiae (lower sequence). Identical sequence positions are indicated in each case between the two sequences. Similar sequence positions are labeled with “+”.

FIG. 3 shows an alignment between an amino acid part-sequence of the invention (corresponding to the complementary strand to position 117 to 1 in SEQ ID NO: 12) (upper sequence) and a part-sequence of the MIPS tag Ade1 from S. cerevisiae (lower sequence). Identical sequence positions are indicated between the two sequences. Similar sequence positions are labeled with “+”.

FIG. 4 shows an alignment between an amino acid part-sequence of the invention (corresponding to the strand of position 1 to 882 in SEQ ID NO: 16) (upper sequence) and a part-sequence of the MIPS tag FRDS1 (2) from S. cerevisiae (lower sequence). Identical sequence positions are indicated between the two sequences. Similar sequence positions are labeled with “+”.

FIG. 5 shows an alignment between an amino acid part-sequence of the invention (corresponding to the complementary strand to position 783 to 1 in SEQ ID NO: 20) (upper sequence) and a part-sequence of the MIPS tag PCK1 from S. cerevisiae (lower sequence). Identical sequence positions are indicated between the two sequences. Similar sequence positions are labeled with “+”.

FIG. 6 shows an alignment between an amino acid part-sequence of the invention (middle sequence) and a part-sequence of the MIPS tag Hem12 from S. cerevisiae (lower sequence). The consensus sequence is depicted above these two. Positions lacking homology are symbolized by black rectangles.

FIG. 7 shows an alignment between an amino acid part-sequence of the invention (corresponding to the complementary strand to position 964 to 529 in SEQ ID NO: 28) (upper sequence) and a part-sequence of the MIPS tag Met1 from S. cerevisiae (lower sequence). Identical sequence positions are indicated between the two sequences. Similar sequence positions are labeled with “+”.

FIG. 8 shows an alignment between an amino acid part-sequence of the invention (corresponding to the strand in position 107 to 313 in SEQ ID NO: 32) (upper sequence) and a part-sequence of the MIPS tag “Hem4” from S. cerevisiae (lower sequence). Identical sequence positions are indicated between the two sequences. Similar sequence positions are labeled with “+”.

FIG. 9 shows an alignment between an amino acid part-sequence of the invention (corresponding to the strand in position 2 to 91 in SEQ ID NO: 36) (upper sequence) and a part-sequence of the MIPS tag Pgk1 from S. cerevisiae (lower sequence). Identical sequence positions are indicated between the two sequences. Similar sequence positions are labeled with “+”.

FIG. 10 shows an alignment between an amino acid part-sequence of the invention (corresponding to the complementary strand to position 713 to 513 in SEQ ID NO: 40) (upper sequence) and a part-sequence of the MIPS tag “PB12” from S. cerevisiae (lower sequence). Identical sequence positions are indicated between the two sequences. Similar sequence positions are labeled with “+”.

FIG. 11 A shows an alignment between an amino acid part-sequence of the invention (corresponding to the complementary strand to position 1596 to 1459 in SEQ ID NO: 44) (upper sequence) and a part-sequence of the MIPS tag CYSK from A. nidulans (lower sequence). FIG. 11B shows an alignment between an amino acid part-sequence of the invention (corresponding to the complementary strand to position 1441 to 971 in SEQ ID NO: 44) (upper sequence) and a part-sequence of the MIPS tag CYSK from A. nidulans (lower sequence). Identical sequence positions are indicated between the two sequences. Similar sequence positions are labeled with “+”.

### DETAILED DESCRIPTION OF THE INVENTION

The nucleic acid molecules of the invention encode polypeptides or proteins which are referred to here as proteins of metabolism (for example with activity in relation to biosynthesis of at least one desired bioproduct or of an intermediate product thereof or to breakdown of byproducts) or for short as “ME proteins”. These ME proteins have, for example, a function in regulating the energy balance of a living system. Owing to the availability of cloning vectors which can be used in Ashbya gossypii, as disclosed, for example, in Wright and Philipsen (1991) Gene, 109, 99-105, and of techniques for genetic manipulation of A. gossypii and the related yeast species, the nucleic acid molecules of the invention can be used for genetic manipulation of these organisms, in particular of A. gossypii, in order to make them better and more efficient producers of vitamin B2 and/or precursors and/or derivatives thereof. This improved production or efficiency may result from a direct effect of the manipulation of a gene of the invention or result from an indirect effect of such a manipulation.

The present invention is based on the provision of novel molecules which are referred to here as ME nucleic acids and ME proteins and are involved in metabolism, in particular in Ashbya gossypii (e.g. in the synthesis or regulation of metabolic enzymes). The activity of the ME molecules of the invention in A. gossypii influences vitamin B2 production by this organism. The activity of the ME molecules of the invention is preferably modulated so that the metabolic and/or energy pathways of A. gossypii in which the ME proteins of the invention are involved are modulated in relation to the yield, production and/or efficiency of vitamin B2 production, thus modulating either directly or indirectly the yield, production and/or efficiency of vitamin B2 production in A. gossypii.

The nucleic acid sequences provided by the invention can be isolated, for example, from the genome of an Ashbya gossypii strain which is freely available from the American Type Culture Collection under the number ATCC 10895.

Improvement in Vitamin B2 Production:

There is a number of possible mechanisms by which the yield, production and/or efficiency of production of vitamin B2 by an A. gossypii strain can be influenced directly through changing the amount and/or activity of a ME protein of the invention.

Thus, it is possible by specific control of metabolism for the efficiency of production of a desired product (e.g. a fine chemical) to be increased or optimized in relation to competing products. It is also possible to make the cells more robust toward external influences so that the viability and thus the productivity in the fermenter is increased.

Mutagenesis of one or more ME proteins of the invention may also lead to ME proteins with altered (increased or reduced) activities which influence indirectly the production of the required product from A. gossypii. It is possible, for example, with the aid of the ME proteins to stop reactions which proceed in direct competition to an intermediate product of the target compound, or to block the metabolic pathway which is responsible for production of this specific intermediate product, and thus optimize production of the desired target substance. The processes which can be influenced include besides the biosynthesis of the product also the construction of the cell walls, transcription, translation, biosynthesis of compounds which are necessary for the growth and division of cells (e.g. nucleotides, amino acids, vitamins, lipids etc.) (Lengeler et al. (1999)). By improving the growth and multiplication of these modified cells it is possible to increase the viability of the cells in cultures on the large scale and also to improve their rate of division so that a comparatively larger number of producing cells can survive in the fermenter culture. The yield, production or efficiency of production can be increased at least because of the presence of a larger number of viable cells each of which produces the required product.

Polypeptides

The invention relates to polypeptides which comprise the abovementioned amino acid sequences or characteristic part-sequences thereof and/or are encoded by the nucleic acid sequences described herein.

The invention likewise encompasses “functional equivalents” of the specifically disclosed novel polypeptides.

“Functional equivalents” or analogs of the specifically disclosed polypeptides are for the purposes of the present invention polypeptides which differ therefrom but which still have the desired biological activity (such as, for example, substrate specificity).

“Functional equivalents” mean according to the invention in particular mutants which have in at least one of the abovementioned sequence positions an amino acid which differs from that specifically mentioned but nevertheless have one of the abovementioned biological activities. “Functional equivalents” thus comprise the mutants obtainable by one or more amino acid additions, substitutions, deletions and/or inversions, it being possible for said modifications to occur in any sequence position as long as they lead to a mutant having the profile of properties of the invention. Functional equivalence exists in particular also when there is qualitative agreement between mutant and unmodified polypeptide in the reactivity pattern, i.e. there are differences in the rate of conversion of identical substrates, for example.

“Functional equivalents” in the above sense are also precursors of the polypeptides described, and functional derivatives and salts of the polypeptides. The term “salts” means both salts of carboxyl groups and acid addition salts of amino groups in the protein molecules of the invention. Salts of carboxyl groups can be prepared in a manner known per se and comprise inorganic salts such as, for example, sodium, calcium, ammonium, iron and zinc salts, and salts with organic bases such as, for example, amines such as triethanolamine, arginine, lysine, piperidine and the like. Acid addition salts such as, for example, salts with mineral acids such as hydrochloric acid or sulfuric acid and salts with organic acids such as acetic acid and oxalic acid are also an aspect of the invention.

“Functional derivatives” of polypeptides of the invention can also be prepared at functional amino acid side groups or at their N- or C-terminal end by known techniques. Such derivatives include for example aliphatic esters of carboxyl groups, amides of carboxyl groups obtainable by reaction with ammonia or with a primary or secondary amine; N-acyl derivatives of free amino groups prepared by reaction with acyl groups; or O-acyl derivatives of free hydroxyl groups prepared by reaction with acyl groups.

“Functional equivalents” naturally also comprise polypeptides which are obtainable from other organisms, and naturally occurring variants. For example homologous sequence regions can be found by sequence comparison, and equivalent enzymes can be established on the basis of the specific requirements of the invention.

“Functional equivalents” likewise comprise fragments, preferably single domains or sequence motifs, of the polypeptides of the invention, which have, for example, the desired biological function.

“Functional equivalents” are additionally fusion proteins which have one of the abovementioned polypeptide sequences or functional equivalents derived therefrom and at least one other heterologous sequence functionally different therefrom in functional N- or C-terminal linkage (i.e. with negligible mutual impairment of the functions of the parts of the fusion proteins). Nonlimiting examples of such heterologous sequences are, for example, signal peptides, enzymes, immunoglobulins, surface antigens, receptors or receptor ligands.

“Functional equivalents” include according to the invention homologs of the specifically disclosed proteins. These have at least 60%, preferably at least 75%, in particular at least 85%, such as, for example, 90%, 95% or 99%, homology to one of the specifically disclosed sequences, calculated by the algorithm of Pearson and Lipman, Proc. Natl. Acad, Sci. (USA) 85(8), 1988, 2444-2448.

In the case where protein glycosylation is possible, equivalents of the invention include proteins of the type defined above in deglycosylated or glycosylated form, and modified forms obtainable by altering the glycosylation pattern.

Homologs of the proteins or polypeptides of the invention can be generated by mutagenesis, for example by point mutation or truncation of the protein. The term “homolog” as used here relates to a variant form of the protein which acts as agonist or antagonist of the protein activity.

Homologs of the proteins of the invention can be identified by screening combinatorial libraries of mutants such as, for example, truncation mutants. It is possible, for example, to generate a variegated library of protein variants by combinatorial mutagenesis at the nucleic acid level, such as, for example, by enzymatic ligation of a mixture of synthetic oligonucleotides. There is a large number of methods which can be used to produce libraries of potential homologs from a degenerate oligonucleotide sequence. Chemical synthesis of a degenerate gene sequence can be carried out in an automatic DNA synthesizer, and the synthetic gene can then be ligated into a suitable expression vector. The use of a degenerate set of genes makes it possible to provide a mixture comprising all sequences which encode the desired set of potential protein sequences. Methods for synthesizing degenerate oligonucleotides are known to the skilled worker (for example Narang, S. A. (1983) Tetrahedron 39:3; Itakura et al. (1984) Annu. Rev. Biochem. 53:323; Itakura et al., (1984) Science 198:1056; Ike et al. (1983) Nucleic Acids Res. 11:477).

In addition, libraries of fragments of the protein codon can be used to generate a variegated population of protein fragments for screening and for subsequent selection of homologs of a protein of the invention. In one embodiment, a library of coding sequence fragments can be generated by treating a double-stranded PCR fragment of a coding sequence with a nuclease under conditions under which nicking takes place only about once per molecule, denaturing the double-stranded DNA, renaturing the DNA to form double-stranded DNA, which may comprise sense/antisense pairs of different nicked products, removing single-stranded sections from newly formed duplices by treatment with S1 nuclease and ligating the resulting fragment library into an expression vector. It is possible by this method to derive an expression library which encodes N-terminal, C-terminal and internal fragments having different sizes of the protein of the invention.

Several techniques are known in the prior art for screening gene products from combinatorial libraries which have been produced by point mutations or truncation and for screening cDNA libraries for gene products with a selected property. These techniques can be adapted to rapid screening of gene libraries which have been generated by combinatorial mutagenesis of homologs of the invention. The most frequently used techniques for screening large gene libraries undergoing high-throughput analysis comprise the cloning of the gene library into replicable expression vectors, transformation of suitable cells with the resulting vector library and expression of the combinatorial genes under conditions under which detection of the required activity facilitates isolation of the vector which encodes the gene whose product has been detected. Recursive ensemble mutagenesis (REM), a technique which increases the frequency of functional mutants in the libraries, can be used in combination with the screening tests for identifying homologs (Arkin and Yourvan (1992) PNAS 89:7811-7815; Delgrave et al. (1993) Protein Engineering 6(3):327-331).

Recombinant preparation of polypeptides of the invention is possible (see following sections) or they can be isolated in native form from microorganisms, especially those of the genus Ashbya, by use of conventional biochemical techniques (see Cooper, T. G., Biochemische Arbeitsmethoden, Verlag Walter de Gruyter, Berlin, New York or in Scopes, R., Protein Purification, Springer Verlag, New York, Heidelberg, Berlin).

Nucleic Acid Sequences:

The invention also relates to nucleic acid sequences (single- and double-stranded DNA and RNA sequences such as, for example, cDNA and mRNA), coding for one of the above polypeptides and their functional equivalents which are obtainable, for example, by use of artificial nucleotide analogs.

The invention relates both to isolated nucleic acid molecules which code. for polypeptides or proteins of the invention or biologically active sections thereof, and to nucleic acid fragments which can be used, for example, for use as hybridization probes or primers for identifying or amplifying coding nucleic acids of the invention.

The nucleic acid molecules of the invention may additionally comprise untranslated sequences from the 3′ and/or 5′ end of the coding region of the gene.

An “isolated” nucleic acid molecule is separated from other nucleic acid molecules which are present in the natural source of the nucleic acid and may moreover be essentially free of other cellular material or culture medium if it is produced by recombinant techniques, or free of chemical precursors or other chemicals if it is chemically synthesized.

A nucleic acid molecule of the invention can be isolated by using standard techniques of molecular biology and the sequence information provided according to the invention. For example, cDNA can be isolated from a suitable cDNA library by using one of the specifically disclosed complete sequences or a section thereof as hybridization probe and standard hybridization techniques (as described, for example, in Sambrook, J., Fritsch, E. F. and Maniatis, T., Molecular Cloning: A Laboratory Manual, 2nd edition, Cold Spring Harbor Laboratory, Cold Spring Harbor Laboratory Press, Cold Spring Harbor, N.Y., 1989). It is moreover possible for a nucleic acid molecule comprising one of the disclosed sequences or a section thereof to be isolated by polymerase chain reaction using the oligonucleotide primers constructed on the basis of this sequence. The nucleic acid amplified in this way can be cloned into a suitable vector and be characterized by DNA sequence analysis. The oligonucleotides of the invention which correspond to an ME nucleotide sequence can also be produced by standard synthetic methods, for example using an automatic DNA synthesizer.

The invention additionally comprises the nucleic acid molecules which are complementary to the specifically described nucleotide sequences, or a section thereof.

The nucleotide sequences of the invention make it possible to generate probes and primers which can be used for identifying and/or cloning homologous sequences in other cell types and organisms. Such probes and primers usually comprise a nucleotide sequence region which hybridizes under stringent conditions onto at least about 12, preferably at least about 25, such as, for example, 40, 50 or 75, consecutive nucleotides of a sense strand of a nucleic acid sequence of the invention or of a corresponding antisense strand.

Further nucleic acid sequences of the invention are derived from SEQ ID NO: 1, 4, 6, 9, 12, 14,16,18, 20, 24, 26, 28, 30, 32, 34, 36, 38, 40, 42, 44 or SEQ ID NO: 47 and differ therefrom through addition, substitution, insertion or deletion of one or more nucleotides, but still code for polypeptides having the desired profile of properties.

The invention also encompasses nucleic acid sequences which comprise so-called silent mutations or are modified, by comparison with a specifically mentioned sequence, in accordance with the codon usage of a specific source or host organism, as well as naturally occurring variants, such as, for example, splice variants or allelic variants, thereof. It likewise relates to sequences which are obtainable by conservative nucleotide substitutions (i.e. the relevant amino acid is replaced by an amino acid with the same charge, size, polarity and/or solubility).

The invention also relates to molecules derived from the specifically disclosed nucleic acids through sequence polymorphisms. These genetic polymorphisms may exist because of the natural variation between individuals within a population. These natural variations normally result in a variance of from 1 to 5% in the nucleotide sequence of a gene.

The invention additionally encompasses nucleic acid sequences which hybridize with or are complementary to the abovementioned coding sequences. These polynucleotides can be found on screening of genomic or cDNA libraries and, where appropriate, be amplified therefrom by means of PCR using suitable primers, and then, for example, be isolated with suitable probes. Another possibility is to transform suitable microorganisms with polynucleotides or vectors of the invention, multiply the microorganisms and thus the polynucleotides, and then isolate them. An additional possibility is to synthesize polynucleotides of the invention by chemical routes.

The property of being able to “hybridize” onto polynucleotides means the ability of a polynucleotide or oligonucleotide to bind under stringent conditions to an almost complementary sequence, while there are no nonspecific bindings between noncomplementary partners under these conditions. For this purpose, the sequences should be 70-100%, preferably 90-100%, complementary. The property of complementary sequences being able to bind specifically to one another is made use of, for example, in the Northern or Southern blot technique or in PCR or RT-PCR in the case of primer binding. Oligonucleotides with a length of 30 base pairs or more are normally employed for this purpose. Stringent conditions mean, for example, in the Northern blot technique the use of a washing solution at 50-70° C., preferably 60-65° C., for example 0.1× SSC buffer with 0.1% SDS (20×SSC: 3M NaCl, 0.3M Na citrate, pH 7.0) for eluting nonspecifically hybridized cDNA probes or oligonucleotides. In this case, as mentioned above, only nucleic acids with a high degree of complementarity remain bound to one another. The setting up of stringent conditions is known to the skilled worker and is described, for example, in Ausubel et al., Current Protocols in Molecular Biology, John Wiley & Sons, N.Y. (1989), 6.3.1-6.3.6.

A further aspect of the invention relates to antisense nucleic acids. This comprises a nucleotide sequence which is complementary to a coding sense nucleic acid. The antisense nucleic acid may be complementary to the entire coding strand or only to a section thereof. In a further embodiment, the antisense nucleic acid molecule is antisense to a noncoding region of the coding strand of a nucleotide sequence. The term “noncoding region” relates to the sequence sections which are referred to as 5′- and 3′-untranslated regions.

An antisense oligonucleotide may be, for example, about 5, 10, 15, 20, 25, 30, 35, 40, 45 or 50 nucleotides long. An antisense nucleic acid of the invention can be constructed by chemical synthesis and enzymatic ligation reactions using methods known in the art.

An antisense nucleic acid can be synthesized chemically, using naturally occurring nucleotides or variously modified nucleotides which are configured so that they increase the biological stability of the molecules or increase the physical stability of the duplex formed between the antisense and sense nucleic acids. Examples which can be used are phosphorothioate derivatives and acridine-substituted nucleotides. Examples of modified nucleosides which can be used for generating the antisense nucleic acid are, inter alia, 5-fluorouracil, 5-bromouracil, 5-chlorouracil, 5-iodouracil, hypoxanthine, xanthine, 4-acetylcytosine, 5-(carboxyhydroxymethyl)uracil, 5-carboxymethylamino-methyl-2-thiouridine, 5-carboxymethylaminomethyluracil, dihydrouracil, beta-D-galacto-sylqueuosine, inosine, N6-isopentenyladenine, 1-methylguanine, 1-methylinosine, 2,2-dimethylguanine, 2-methyladenine, 2-methylguanine, 3-methylcytosine, 5-methyl-cytosine, N6-methyladenine, 7-methylguanine, 5-methylaminomethyluracil, 5-methoxy-aminomethyl-2-thiouracil, beta-D-mannosylqueuosine, 5-methoxycarboxymethyluracil, 5-methoxyuracil, 2-methylthio-N6-isopentenyladenine, uracil-5-oxyacetic acid (v), wybutoxosine, pseudouracil, queuosine, 2-thiocytosine, 5-methyl-2-thiouracil, 2-thio-uracil, 4-thiouracil, 5-methyluracil, methyl uracil-5-oxyacetate, 3-(3-amino-3-carboxy-propyl)uracil, (acp3)w and 2,6-diaminopurine. The antisense nucleic acid may also be produced biologically by using an expression vector into which a nucleic acid has been subcloned in the antisense direction.

The antisense nucleic acid molecules of the invention are normally administered to a cell or generated in situ so that they hybridize with the cellular mRNA and/or a coding DNA or bind thereto, so that expression of the protein is inhibited for example by inhibition of transcription and/or translation.

The antisense molecule can be modified so that it binds specifically to a receptor or to an antigen which is expressed on a selected cell surface, for example through linkage of the antisense nucleic acid molecule to a peptide or an antibody which binds to a cell surface receptor or antigen. The antisense nucleic acid molecule can also be administered to cells by using the vectors described herein. The vector constructs preferred for achieving adequate intracellular concentrations of the antisense molecules are those in which the antisense nucleic acid molecule is under the control of a strong bacterial, viral or eukaryotic promoter.

In a further embodiment, the antisense nucleic acid molecule of the invention is an alpha-anomeric nucleic acid molecule. An alpha-anomeric nucleic acid molecule forms specific double-stranded hybrids with complementary RNA, with the strands running parallel to one another, in contrast to normal alpha units (Gaultier et al., (1987) Nucleic Acids Res. 15:6625-6641). The antisense nucleic acid molecule may additionally comprise a 2′-O-methylribonucleotide (Inoue et al., (1987) Nucleic Acids Res. 15:6131-6148) or a -chimeric RNA-DNA analog (Inoue et al. (1987) FEBS Lett. 215:327-330).

The invention also relates to ribozymes. These are catalytic RNA molecules with ribonuclease activity which are able to cleave a single-stranded nucleic acid such as an mRNA to which they have a complementary region. It is thus possible to use ribozymes (for example hammerhead ribozymes (described in Haselhoff and Gerlach (1988) Nature 334:585-591)) for the catalytic cleavage of transcripts of the invention in order thereby to inhibit the translation of the corresponding nucleic acid. A ribozyme with specificity for a coding nucleic acid of the invention can be formed, for example, on the basis of a cDNA specifically disclosed herein. For example a derivative of a tetrahymena-L-19 IVS RNA can be constructed, with the nucleotide sequence of the active site being complementary to the nucleotide sequence to be cleaved in a coding mRNA of the invention. (Compare, for example, US-A-4 987 071 and US-A-5 116 742). Alternatively, mRNA can be used for selecting a catalytic RNA with specific ribonuclease activity from a pool of RNA molecules (see, for example, Bartel, D., and Szostak, J. W. (1993) Science 261:1411-1418).

Gene expression of sequences of the invention can alternatively be inhibited by targeting nucleotide sequences which are complementary to the regulatory region of a nucleotide sequence of the invention (for example to a promoter and/or enhancer of a coding sequence) so that there is formation of triple helix structures which prevent transcription of the corresponding gene in target cells (Helene, C. (1991) Anticancer Drug Res. 6(6) 569-584; Helene, C. et al., (1992) Ann. N. Y. Acad. Sci. 660:27-36; and Maher., L.J. (1992) Bioassays 14(12):807-815).

Expression Constructs and Vectors:

The invention additionally relates to expression constructs comprising, under the genetic control of regulatory nucleic acid sequences, a nucleic acid sequence coding for a polypeptide of the invention; and to vectors comprising at least one of these expression constructs. Such constructs of the invention preferably comprise a promoter 5′-upstream from the particular coding sequence, and a terminator sequence 3′-downstream, and, where appropriate, other usual regulatory elements, in particular each operatively linked to the coding sequence. “Operative linkage” means the sequential arrangement of promoter, coding sequence, terminator and, where appropriate, other regulatory elements in such a way that each of the regulatory elements is able to comply with its function as intended for expression of the coding sequence. Examples of sequences which can be operatively linked are targeting sequences and enhancers, polyadenylation signals and the like. Other regulatory elements comprise selectable markers, amplification signals, origins of replication and the like. Suitable regulatory sequences are described, for example, in Goeddel, Gene Expression Technology: Methods in Enzymology 185, Academic Press, San Diego, Calif. (1990).

In addition to the artificial regulatory sequences it is possible for the natural regulatory sequence still to be present in front of the actual structural gene. This natural regulation can, where appropriate, be switched off by genetic modification, and expression of the genes can be increased or decreased. The gene construct can, however, also have a simpler structure, that is to say no additional regulatory signals are inserted in front of the structural gene, and the natural promoter with its regulation is not deleted. Instead, the natural regulatory sequence is mutated so that regulation no longer takes place, and gene expression is enhanced or diminished. The nucleic acid sequences may be present in one or more copies in the gene construct.

Examples of promoters which can be used are: cos, tac, trp, tet, trp-tet, Ipp, lac, Ipp-lac, laclq, T7, T5, T3, gal, trc, ara, SP6, λ-PR or λ-PL promoter, which are advantageously used in Gram-negative bacteria; and the Gram-positive promoters any and SPO2, the yeast promoters ADC1, MFα, AC, P-60, CYC1, GAPDH or the plant promoters CaMW35S, SSU, OCS, lib4, usp, STLS1, B33, not or the ubiquitin or phaseolin promoter. The use of inducible promoters is particularly preferred, such as, for example, light- and, in particular, temperature-inducible promoters such as the PᵣPₗ promoter. It is possible in principle for all natural promoters with their regulatory sequences to be used. In addition, it is also possible advantageously to use synthetic promoters.

Said regulatory sequences are intended to make specific expression of the nucleic acid sequences possible. This may mean, for example, depending on the host organism, that the gene is expressed or overexpressed only after induction or that it is immediately expressed and/or overexpressed.

The regulatory sequences or factors may moreover preferably influence positively, and thus increase or reduce, expression. Thus, enhancement of the regulatory elements can take place advantageously at the level of transcription by using strong transcription signals such as promoters and/or enhancers. However, it is also possible to enhance translation by, for example, improving the stability of the mRNA.

An expression cassette is produced by fusing a suitable promoter to a suitable nucleotide sequence according to the invention and to a terminator signal or polyadenylation signal. Conventional techniques of recombination and cloning are used for this purpose, as described, for example, in T. Maniatis, E. F. Fritsch and J. Sambrook, Molecular Cloning: A Laboratory Manual, Cold Spring Harbor Laboratory, Cold Spring Harbor, N.Y. (1982) and in T. J. Silhavy, M. L. Berman and L. W. Enquist, Experiments with Gene Fusions, Cold Spring Harbor Laboratory, Cold Spring Harbor, N.Y. (1984) and in Ausubel, F. M. et al., Current Protocols in Molecular Biology, Greene Publishing Assoc. and Wiley Interscience (1987).

For expression in a suitable host organism, the recombinant nucleic acid construct or gene construct is advantageously inserted into a host-specific vector, which makes optimal expression of the genes in the host possible. Vectors are well known to the skilled worker and can be found, for example, in “Cloning Vectors” (Pouwels P. H. et al., eds, Elsevier, Amsterdam-New York-Oxford, 1985). Vectors also mean not only plasmids but also all other vectors known to the skilled worker, such as, for example, phages, viruses, such as SV40, CMV, baculovirus and adenovirus, transposons, IS elements, phasmids, cosmids, and linear or circular DNA. These vectors may undergo autonomous replication in the host organism or chromosomal replication.

Examples of suitable expression vectors which may be mentioned are:

Conventional fusion expression vectors such as pGEX (Pharmacia Biotech Inc; Smith, D. B. and Johnson, K. S. (1988) Gene 67:3140), pMAL (New England Biolabs, Beverly, Mass.) and pRIT 5 (Pharmacia, Piscataway, N.J.), with which respectively glutathione S-transferase (GST), maltose E-binding protein and protein A are fused to the recombinant target protein.

Non-fusion protein expression vectors such as pTrc (Amann et al., (1988) Gene 69:301-315) and pET 11 d (Studier et al. Gene Expression Technology: Methods in Enzymology 185, Academic Press, San Diego, Calif. (1990) 60-89).

Yeast expression vector for expression in the yeast S. cerevisiae, such as pYepSec1(Baldari et al., (1987) Embo J. 6:229-234), pMF (Kurjan and Herskowitz (1982) Cell 30:933-943), pJRY88 (Schultz et al. (1987) Gene 54:113-123) and pYES2 (Invitrogen Corporation, San Diego, Calif.). Vectors and methods for constructing vectors suitable for the use in other fungi such as filamentous fungi comprise those which are described in detail in: van den Hondel, C. A. M. J. J. & Punt, P. J. (1991) “Gene transfer systems and vector development for filamentous fungi” in: Applied Molecular Genetics of Fungi, J. F. Peberdy et al., eds, pp. 1-28, Cambridge University Press: Cambridge.

Baculovirus vectors which are available for expression of proteins in cultured insect cells (for example Sf9 cells) comprise the pAc series (Smith et al., (1983) Mol. Cell Biol.. 3:2156-2165) and pVL series (Lucklow and Summers (1989) Virology 170:31-39).

Plant expression vectors such as those described in detail in: Becker, D., Kemper, E., Schell, J. and Masterson, R. (1992) “New plant binary vectors with selectable markers located proximal to the left border”, Plant Mol. Biol. 20:1195-1197; and Bevan, M. W. (1984) “Binary Agrobacterium vectors for plant transformation”, Nucl. Acids Res. 12:8711-8721.

Mammalian expression vectors such as pCDM8 (Seed, B. (1987) Nature 329:840) and pMT2PC (Kaufman et al. (1987) EMBO J. 6:187-195).

Further suitable expression systems for prokaryotic and eukaryotic cells are described in chapters 16 and 17 of Sambrook, J., Fritsch, E. F. and Maniatis, T., Molecular cloning: A Laboratory Manual, 2nd edition, Cold Spring Harbor Laboratory, Cold Spring Harbor Laboratory Press, Cold Spring Harbor, N.Y., 1989.

Recombinant Microorganisms:

The vectors of the invention can be used to produce recombinant microorganisms which are transformed, for example, with at least one vector of the invention and can be employed for producing the polypeptides of the invention. The recombinant constructs of the invention described above are advantageously introduced and expressed in a suitable host system. Cloning and transfection methods familiar to the skilled worker, such as, for example, coprecipitation, protoplast fusion, electroporation, retroviral transfection and the like, are preferably used to bring about expression of said nucleic acids in the particular expression system. Suitable systems are described, for example, in Current Protocols in Molecular Biology, F. Ausubel et al., eds, Wiley Interscience, New York 1997, or Sambrook et al. Molecular Cloning: A Laboratory Manual, 2nd edition, Cold Spring Harbor Laboratory, Cold Spring Harbor Laboratory Press, Cold Spring Harbor, N.Y., 1989.

It is also possible according to the invention to produce homologously recombined microorganisms. This entails production of a vector which contains at least one section of a gene of the invention or a coding sequence, in which, where appropriate, at least one amino acid deletion, addition or substitution has been introduced in order to modify, for example functionally disrupt, the sequence of the invention (knockout vector). The introduced sequence may, for example, also be a homolog from a related microorganism or be derived from a mammalian, yeast or insect source. The vector used for homologous recombination may alternatively be designed so that the endogenous gene is mutated or otherwise modified during the homologous recombination but still encodes the functional protein (for example the regulatory region located upstream may be modified in such a way as to modify expression of the endogenous protein). The modified section of the ME gene is in the homologous recombination vector. The construction of suitable vectors for homologous recombination is, for example, described in Thomas, K. R. and Capecchi, M. R. (1987) Cell 51:503.

Suitable host organisms are in principle all organisms which enable expression of the nucleic acids of the invention, or of their allelic variants, or their functional equivalents or derivatives. Host organisms mean, for example, bacteria, fungi, yeasts, plant or animal cells. Preferred organisms are bacteria, such as those of the genera Escherichia, such as, for example, Escherichia coli, Streptomyces, Bacillus or Pseudomonas, eukaryotic microorganisms such as Saccharomyces cerevisiae, Aspergillus, higher eukaryotic cells from animals or plants, for example Sf9 or CHO cells. Preferred organisms are selected from the genus Ashbya, in particular from A. gossypii strains.

Successfully transformed organisms can be selected through marker genes which are likewise present in the vector or in the expression cassette. Examples of such marker genes are genes for antibiotic resistance and for enzymes which catalyze a color-forming reaction which causes staining of the transformed cell. These can then be selected by automatic cell sorting. Microorganisms which have been successfully transformed with a vector and harbor an appropriate antibiotic resistance gene (for example G418 or hygromycin) can be selected by appropriate antibiotic-containing media or nutrient media. Marker proteins present on the surface of the cell can be used for selection by means of affinity chromatography.

The combination of the host organisms and the vectors appropriate for the organisms, such as plasmids, viruses or phages, such as, for example, plasmids with the RNA polymerase/promoter system, phages λ or μ or other temperate phages or transposons and/or other advantageous regulatory sequences forms an expression system. The term “expression system” means, for example, the combination of mammalian cells, such as CHO cells, and vectors, such as pcDNA3neo vector, which are suitable for mammalian cells.

If desired, the gene product can also be expressed in transgenic organisms such as transgenic animals such as, in particular, mice, sheep or transgenic plants.

Recombinant Production of the Polypeptides:

The invention further relates to methods for the recombinant production of a polypeptide of the invention or functional, biologically active fragments thereof, where a polypeptide-producing microorganism is cultured, expression of the polypeptides is induced where appropriate, and they are isolated from the culture. The polypeptides can also be produced on the industrial scale in this way if desired.

The recombinant microorganism can be cultured and fermented by known methods. Bacteria can be grown, for example, in TB or LB medium and at a temperature of 20 to 40° C. and a pH of from 6 to 9. Details of suitable culturing conditions are described, for example, in T. Maniatis, E. F. Fritsch and J. Sambrook, Molecular Cloning: A Laboratory Manual, Cold Spring Harbor Laboratory, Cold Spring Harbor, N.Y. (1989).

If the polypeptides are not secreted into the culture medium, the cells are then disrupted and the product is obtained from the lysate by known protein isolation methods. The cells may alternatively be disrupted by high-frequency ultrasound, by high pressure, such as, for example, in a French pressure cell, by osmolysis, by the action of detergents, lytic enzymes or organic solvents, by homogenizers or by a combination of a plurality of the methods mentioned.

The polypeptides can be purified by known chromatographic methods such as molecular sieve chromatography (gel filtration), such as Q-Sepharose chromatography, ion exchange chromatography and hydrophobic chromatography, and by other usual methods such as ultrafiltration, crystallization, salting out, dialysis and native gel electrophoresis. Suitable methods are described, for example, in Cooper, T. G., Biochemische Arbeitsmethoden, Verlag Walter de Gruyter, Berlin, New York or in Scopes, R., Protein Purification, Springer Verlag, New York, Heidelberg, Berlin.

It is particularly advantageous for isolation of the recombinant protein to use vector systems or oligonucleotides which extend the cDNA by particular nucleotide sequences and thus code for modified polypeptides or fusion proteins which serve, for example, for simpler purification. Suitable modifications of this type are, for example, so-called tags which act as anchors, such as, for example, the modification known as hexa-histidine anchor, or epitopes which can be recognized as antigens by antibodies (described, for example, in Harlow, E. and Lane, D., 1988, Antibodies: A Laboratory Manual, Cold Spring Harbor (N.Y.) Press). These anchors can be used to attach the proteins to a solid support, such as, for example, a polymer matrix, which can, for example, be packed into a chromatography column, or can be used on a microtiter plate or another support.

These anchors can at the same time also be used for recognition of the proteins. It is also possible to use for recognition of the proteins conventional markers such as fluorescent dyes, enzyme markers which form a detectable reaction product after reaction with a substrate, or radioactive labels, alone or in combination with the anchors for derivatizing the proteins.

The invention additionally relates to a method for the microbiological production of vitamin B2 and/or precursors and/or derivatives thereof.

If the conversion is carried out with a recombinant microorganism, the microorganisms are preferably initially cultured in the presence of oxygen and in a complex medium, such as, for example, at a culturing temperature of about 20° C. or more, and at a pH of about 6 to 9 until an adequate cell density is reached. In order to be able to control the reaction better, it is preferred to use an inducible promoter. The culturing is continued in the presence of oxygen for 12 hours to 3 days after induction of vitamin B2 production.

The following nonlimiting examples describe specific embodiments of the invention.

General Experimental Details

a) General cloning methods

The cloning steps carried out for the purpose of the present invention, such as, for example, restriction cleavages, agarose gel electrophoresis, purification of DNA fragments, transfer of nucleic acids to nitrocellulose and nylon membranes, linkage of DNA fragments, transformation of E. coli cells, culturing of bacteria, replication of phages and sequence analysis of recombinant DNA, were carried out as described by Sambrook et al. (1989) loc. cit.

b) Polymerase chain reaction (PCR)

PCR was carried out in accordance with a standard protocol with the following standard mixture:

8 μl of dNTP mix (200 μM), 10 μl of Taq polymerase buffer (10×) without MgCl₂, 8 μl of MgCl₂ (25 mM), 1 μl of each primer (0.1 μM), 1 μl of DNA to be amplified, 2.5 U of Taq polymerase (MBI Fermentas, Vilnius, Lithuania), demineralized water ad 100 μl .

c) Culturing of E.coli

The recombinant E. coli DH5α strain was cultured in LB-amp medium (tryptone 10.0 g, NaCl 5.0 g, yeast extract 5.0 g, ampicillin 100 g/ml, H₂0 ad 1000 ml) at 37° C. For this purpose, in each case one colony was transferred, using an inoculating loop, from an agar plate into 5 ml of LB-amp. After culturing for about 18 hours shaking at a frequency of 220 rpm, 400 ml of medium in a 2 I flask were inoculated with 4 ml of culture. Induction of P450 expression in E. coli took place after the OD578 reached a value between 0.8 and 1.0 by heat-shock induction at 42° C. for three to four hours.

d) Purification of the required product from the culture

The required product can be isolated from the microorganism or from the culture supernatant by various methods known in the art. If the required product is not secreted by the cells, the cells can be harvested from the culture by slow centrifugation, and the cells can be lysed by standard techniques such as mechanical force or ultrasound treatment.

The cell detritus is removed by centrifugation, and the supernatant fraction which contains the soluble proteins is obtained for further purification of the required compound. If the product is secreted by the cells, the cells are removed from the culture by slow centrifugation, and the supernatant fraction is retained for further purification.

The supernatant fraction from the two purification methods is subjected to chromatography with a suitable resin, with the required molecule either being retained on the chromatography resin, or passing through the latter, with greater selectivity than the impurities. These chromatography steps can be repeated if necessary, using the same or different chromatography resins. The skilled worker is proficient in the selection of suitable chromatography resins and their most effective use for a particular molecule to be purified. The purified product can be concentrated by filtration or ultrafiltration and be stored at a temperature at which the stability of the product is maximal.

Many purification methods are known in the art. These purification techniques are described, for example, in Bailey, J. E. & Ollis, D. F. Biochemical Engineering Fundamentals, McGraw-Hill: New York (1986).

The identity and purity of the isolated compounds can be determined by prior art techniques. These comprise high performance liquid chromatography (HPLC), spectroscopic methods, staining methods, thin layer chromatography, NIRS, enzyme assay or microbiological assays. These analytical methods are summarized in: Patek et al. (1994) Appl. Environ. Microbiol. 60:133-140; Malakhova et al. (1996) Biotekhnologiya 11 27-32; and Schmidt et al. (1998) Bioprocess Engineer, 19:67-70. Ullmann's Encyclopedia of Industrial Chemistry (1996) Vol. A27, VCH: Weinheim, pp. 89-90, pp. 521-540, pp. 540-547, pp. 559-566, pp. 575-581 and pp. 581-587; Michal, G (1999) Biochemical Pathways: An Atlas of Biochemistry and Molecular Biology, John Wiley and Sons; Fallon, A. et al. (1987) Applications of HPLC in Biochemistry in: Laboratory Techniques in Biochemistry and Molecular Biology, Vol.17.

e) General description of the MPSS method, clone identification and homology search

The MPSS technology (Massive Parallel Signature Sequencing as described by Brenner et al, Nat. Biotechnol. (2000) 18, 630-634; to which express reference is hereby made) was applied to the filamentous, vitamin B2-producing fungus Ashbya gossypii. It is possible with the aid of this technology to obtain with high accuracy quantitative information about the level of expression of a large number of genes in a eukaryotic organism. This entails the mRNA of the organism being isolated at a particular time X, being transcribed with the aid of the enzyme reverse transcriptase into cDNA and then being cloned into special vectors which have a specific tag sequence. The number of vectors with a different tag sequence is chosen to be high enough (about 1000 times higher) for statistically each DNA molecule to be cloned into a vector which is unique through its tag sequence.

The vector inserts are then cut out together with the tag. The DNA molecules obtained in this way are then incubated with microbeads which possess the molecular counterparts of the tags mentioned. After incubation it can be assumed that each microbead is loaded via the specific tags or counterparts with only one type of DNA molecules. The beads are transferred into a special flow cell and fixed there so that it is possible to carry out a mass sequencing of all the beads with the aid of an adapted sequencing method based on fluorescent dyes and with the aid of a digital color camera. Although numerically high analysis is possible with this method, it is limited by a reading width of about 16 to 20 base pairs. The sequence length is, however, sufficient to make an unambiguous correlation between sequence and gene possible for most organisms (20 bp have a sequence frequency of ˜1×10¹²; compared with this, the human genome has a size of “only” ˜3×10⁹ bp).

The data obtained in this way are analyzed by counting the number of identical sequences and comparing the frequencies with one another. Frequently occurring sequences reflect a high level of expression, and sequences which occur sporadically a low level of expression. If the mRNA was isolated at two different time points (X and Y), it is possible to construct a chronological expression pattern of individual genes.

### EXAMPLE 1

Isolation of mRNA from Ashbya gossypii

Ashbya gossypii was cultured in a manner known per se (nutrient medium: 27.5 g/l yeast extract; 0.5 g/l magnesium sulfate; 50 ml/l soybean oil; pH 7). Ashbya gossypii mycelium samples are taken at various times during the fermentation (24 h, 48 h and 72 h), and the corresponding RNA or mRNA is isolated therefrom according to the protocol of Sambrook et al. (1989).

### EXAMPLE 2

Application of MPSS

Isolated mRNA from A. gossypii is then subjected to an MPSS analysis as explained above.

The sets of data found are subjected to a statistical analysis and categorized according to the significance of the differences in expression. This entailed examination both in relation to an increase and a reduction in the level of expression. A division is made by classifying the change in expression into a) monotonic change, b) change after 24 h, and c) change after 48 h.

The 20 bp sequences representing a change in expression and found by MPSS analysis are then used as probes and hybridized with a gene library from Ashbya gossypii, with an average insert size of about 1 kb. The hybridization temperature in this case was in the range from about 30 to 57° C.

### EXAMPLE 3

Construction of a Genomic Gene Library From Ashbya gossypii

To construct a genomic DNA library, initially chromosomal DNA is isolated by the method of Wright and Philippsen (Gene (1991) 109: 99-105) and Mohr (1995, PhD Thesis, Biozentrum Universitat Basel, Switzerland).

The DNA is partially digested with Sau3A. For this purpose, 6 μg of genomic DNA are subjected to a Sau3A digestion with various amounts of enzyme (0.1 to 1 U). The fragments are fractionated in a sucrose density gradient. The 1 kb region is isolated and subjected to a QiaEx extraction. The largest fragments are ligated to the BamHI-cut vector pRS416 (Sikorski and Hieter, Genetics (1988) 122; 19-27) (90 ng of BamHI-cut, dephosphorylated vector; 198 ng of insert DNA; 5 ml of water; 2 μl of 10×ligation buffer; 1 U ligase). This ligation mixture is used to transform the E. coli laboratory strain XL 1 blue, and the resulting clones are employed for identifying the insert.

### EXAMPLE 4

Preparation of an Ordered Gene Library (CHIP Technology)

About 25,000 colonies of the Ashbya gossypii gene library (this corresponds to approximately a 3-fold coverage of the genome) were transferred in an ordered manner to a nylon membrane and then treated by the method of colony hybridization as described in Sambrook et al. (1989). Oligonucleotides were synthesized from the 20 bp sequences found by MPSS analysis and were radiolabeled with ³²P. In each case 10 labeled oligonucleotides with a similar melting point are combined and hybridized together with the nylon membranes. After hybridization and washing steps, positive clones are identified by autoradiography and analyzed directly by PCR sequencing.

In this way, a clone which harbors an insert with the internal name “Oligo 72” and has significant homologies with the MIPS tag “Ade3” from S. cerevisiae was identified. The insert has a nucleic acid sequence as shown in SEQ ID NO: 1.

In this way, a further clone which harbors an insert with the internal name “Oligo 81” and has significant homologies with the MIPS tag “Arg1” from S. cerevisiae was identified. The insert has a nucleic acid sequence as shown in SEQ ID NO: 6.

In this way, a further clone which harbors an insert with the internal name “Oligo 86” and has significant homologies with the MIPS tag “Ade1” from S. cerevisiae was identified. The insert has a nucleic acid sequence as shown in SEQ ID NO: 12.

In this way, a further clone which harbors an insert with the internal name “Oligo 162” and has significant homologies with the MIPS tag “FRDS1 (2)” from S. cerevisiae was identified. The insert has a nucleic acid sequence as shown in SEQ ID NO: 16.

In this way, a further clone which harbors an insert with the internal name “Oligo 178” and has significant homologies with the MIPS tag “AgPCK1” from S. cerevisiae was identified. The insert has a nucleic acid sequence as shown in SEQ ID NO: 20.

In this way, a further clone which harbors an insert with the internal name “Oligo 64” and has significant homologies with the MIPS tag “Hem 12” from S. cerevisiae was identified. The insert has a nucleic acid sequence as shown in SEQ ID NO: 24.

In this way, a further clone which harbors an insert with the internal name “Oligo 125” and has significant homologies with the MIPS tag “Met1” from S. cerevisiae was identified. The insert has a nucleic acid sequence as shown in SEQ ID NO: 28.

In this way, a further clone which harbors an insert with the internal name “Oligo 107” and has significant homologies with the MIPS tag “Hem4” from S. cerevisiae was identified. The insert has a nucleic acid sequence as shown in SEQ ID NO: 32.

In this way, a further clone which harbors an insert with the internal name “Oligo 136” and has significant homologies with the MIPS tag “Pgk1” from S. cerevisiae was identified. The insert has a nucleic acid sequence as shown in SEQ ID NO: 36.

In this way, a further clone which harbors an insert with the internal name “Oligo 157” and has significant homologies with the MIPS tag “PB12” from S. cerevisiae was identified. The insert has a nucleic acid sequence as shown in SEQ ID NO: 40.

In this way, a clone which harbors an insert with the internal name “Oligo 108” and has significant homologies with the MIPS tag “CYSK” from A. nidulans was identified. The insert has a nucleic acid sequence as shown in SEQ ID NO: 44.

### EXAMPLE 5

Analysis of the Sequence Data by Means of a BLASTX Search

An analysis of the resulting nucleic acid sequences, i.e. their functional assignment to a functional amino acid sequence took place by means of a BLASTX search in sequence databases. Almost all of the amino acid sequence homologies found related to Saccharomyces cerevisiae (baker's yeast). Since this organism had already been completely sequenced, more detailed information about these genes could be referred to at:

http:/www.mips.gsf.de/proi/veast/search/code search.htm.

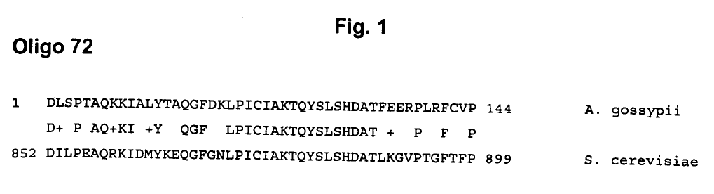

Thus, the following homologies with an amino acid fragment, predominantly from S. cerevisiae, were found. The corresponding alignments are shown in FIGS. 1 to 11, which are appended.

a) The amino acid sequence derived from the coding strand of SEQ ID NO:1 has significant sequence homology with an S. cerevisiae C1-tetrahydrofolate synthase. An amino acid part-sequence derived therefrom (corresponding to nucleotides 1 to 144 from SEQ ID NO:1) with a part-sequence of the S. cerevisiae enzyme is depicted in FIG. 1. A further homology was found for the amino acid part-sequence corresponding to nucleotides 180 to 287 in SEQ ID NO: 1. SEQ ID NO: 2 and SEQ ID No: 3 each show an N-terminally extended amino acid part-sequence.

The A. gossypii nucleic acid sequence found could thus be assigned the function of a C1-tetrahydrofolate synthase.

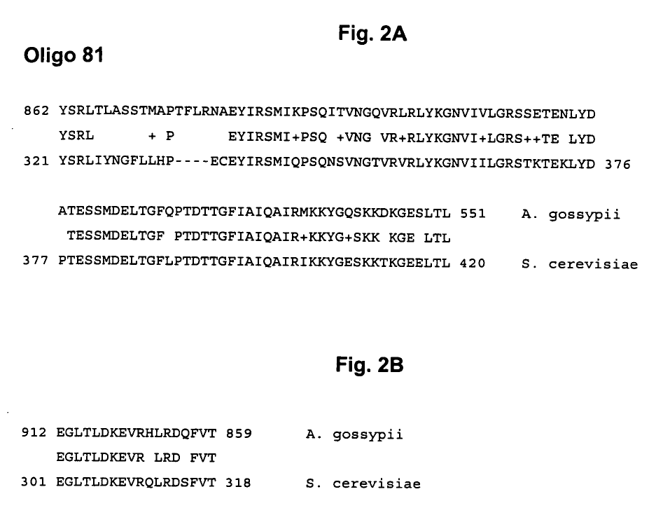

b) The amino acid sequence derived from the corresponding complementary strand to SEQ ID NO: 6 has significant sequence homology with an argininosuccinate synthase from S. cerevisiae. An amino acid part-sequence derived therefrom (corresponding to the complementary strand to nucleotides 862 to 551 from SEQ ID NO: 6) with a part-sequence of the S. cerevisiae enzyme is depicted in FIG. 2A. A further amino acid part-sequence derived therefrom (corresponding to the complementary strand to nucleotides 912 to 859 from SEQ ID NO: 6) with a part-sequence of the S. cerevisiae enzyme is depicted in FIG. 2B. SEQ ID NO: 7 and SEQ ID NO: 8 each show an N-terminally extended amino acid part-sequence.

The A. gossypii nucleic acid sequence found could thus be assigned the function of an argininosuccinate synthase.

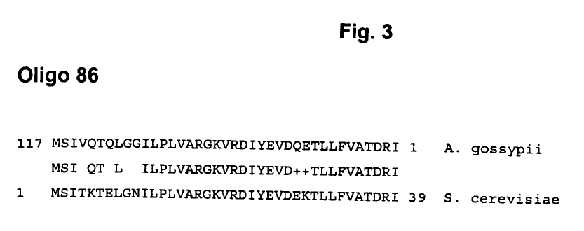

c) The amino acid sequence derived from the corresponding complementary strand to SEQ ID NO: 12 has significant sequence homology with the phosphoribosyl-aminoimidazole-succinocarboxamide synthase from S. cerevisiae. An amino acid part-sequence derived therefreom (corresponding to nucleotides 117 to 1 from SEQ ID NO: 12) with a part-sequence of the S. cerevisiae enzyme is depicted in FIG. 3. SEQ ID NO: 13 shows an N-terminally extended amino acid part-sequence.

The A. gossypii nucleic acid sequence found could thus be assigned the function of a phosphoribosylaminoimidazole-succinocarboxamide synthase.

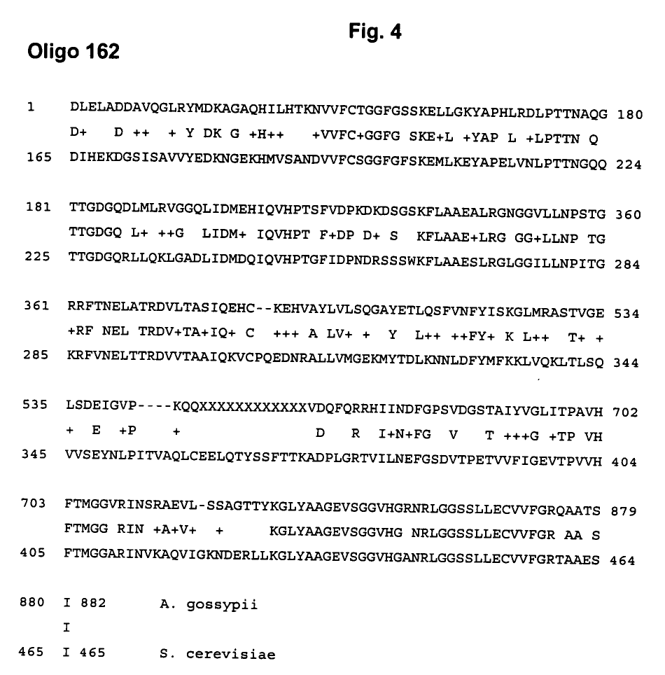

d) The amino acid sequence derived from the coding strand shown in SEQ ID NO: 16 has significant sequence homology with a fumarate reductase from S. cerevisiae. An amino acid part-sequence derived therefrom (corresponding to nucleotides 1 to 882 from SEQ ID NO: 16) with a part-sequence of the S. cerevisiae enzyme is depicted in FIG. 4. SEQ ID NO: 17 shows an N-terminally extended amino acid part-sequence.

The A. gossypii nucleic acid sequence found could thus be assigned the function of a fumarate reductase.

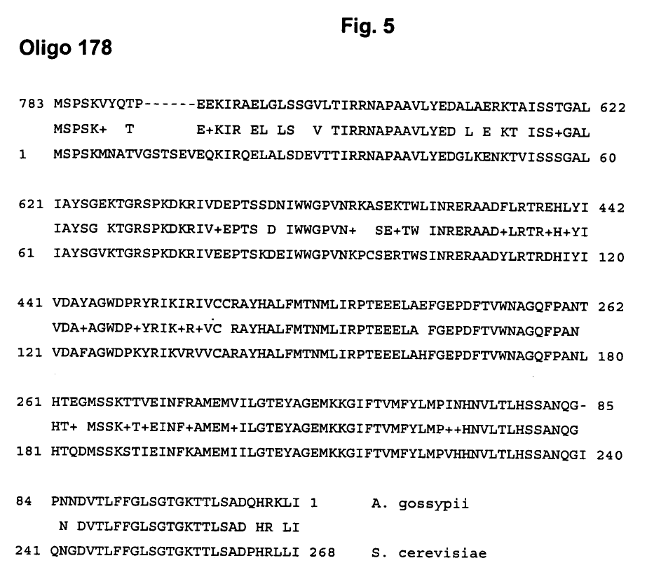

e) The amino acid sequence derived from the corresponding complementary strand to SEQ ID NO: 20 has significant sequence homology with a phosphoenolpyruvate carboxykinase from S. cerevisiae. An amino acid part-sequence derived therefrom (corresponding to nucleotides 783 to 1 from SEQ ID NO: 20) with a part-sequence of the S. cerevisiae enzyme is depicted in FIG. 5. SEQ ID NO: 21 shows an N-terminally extended amino acid part-sequence.

The A. gossypii nucleic acid sequence found could thus be assigned the function of a phosphoenolpyruvate carboxykinase.

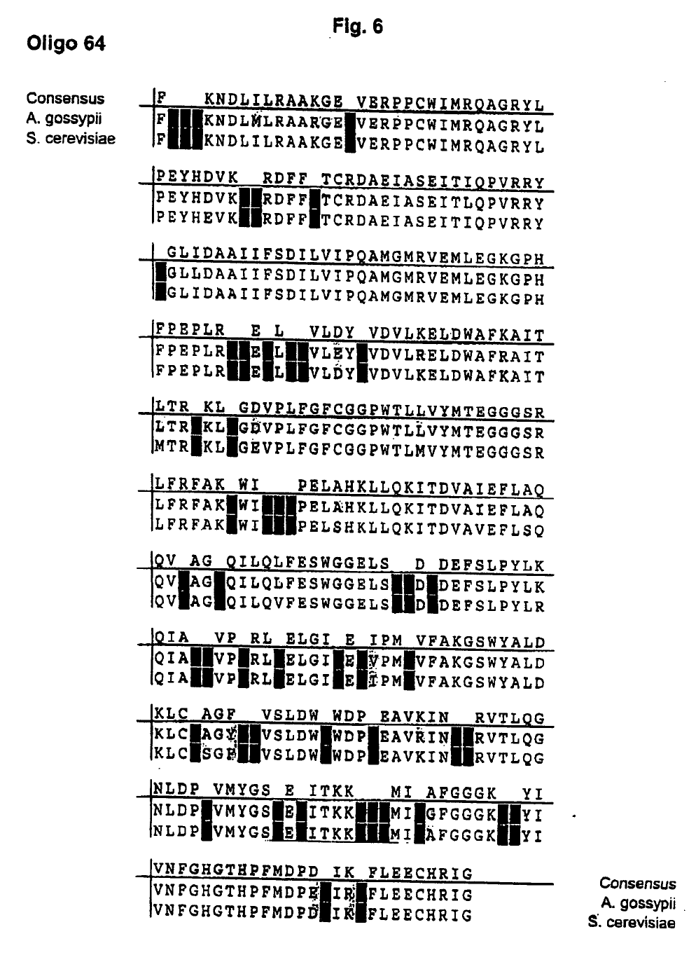

f) The amino acid sequence derived from the coding strand to SEQ ID NO: 24 has significant sequence homology with a uroporphyrinogen decarboxylase from S. cerevisiae. An amino acid part-sequence derived therefrom (SEQ ID NO: 25 corresponding to nucleotides 441 to 1058 from SEQ ID NO: 24) shows homology with a part-sequence of the MIPS tag Hem12 from S. cerevisiae. An amino acid part-sequence derived therefrom with a part-sequence of the S. cerevisiae enzyme is depicted in FIG. 6.

The A. gossypii nucleic acid sequence found could thus be assigned the function of a uroporphyrinogen decarboxylase.

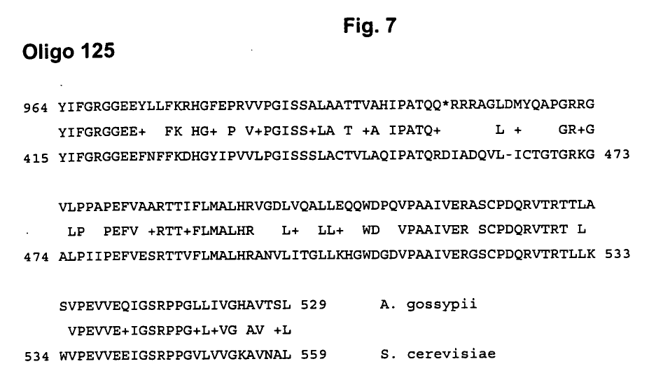

g) The amino acid sequence derived from the complementary strand to SEQ ID NO: 28 has significant sequence homology with a siroheme synthase from S. cerevisiae. An amino acid part-sequence derived therefrom (corresponding to the complementary strand to nucleotides 966 to 529 from SEQ ID NO: 28) with a part-sequence of the S. cerevisiae enzyme is depicted in FIG. 7. SEQ ID NO: 29 shows an N-terminally extended amino acid part-sequence.

The A. gossypii nucleic acid sequence found could thus be assigned the function of a siroheme synthase or of a uroporphyrin III C-methyltransferase.

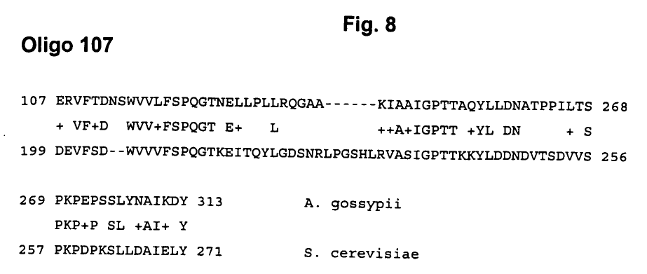

h) The amino acid sequence derived from the coding strand to SEQ ID NO: 32 has significant sequence homology with a uroporphyrinogen III synthase from S. cerevisiae. An amino acid part-sequence derived therefrom (corresponding to nucleotides 107 to 313 from SEQ ID NO: 32) with a part-sequence of the S. cerevisiae enzyme is depicted in FIG. 8. SEQ ID NO: 33 shows an N-terminally extended amino acid part-sequence.

The A. gossypii nucleic acid sequence found could thus be assigned the function of a uroporphyrinogen III synthase.

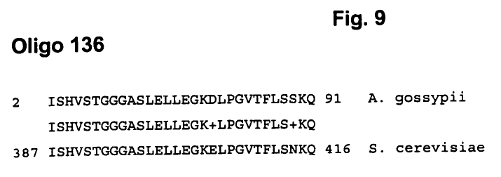

i) The amino acid sequence derived from the coding strand to SEQ ID NO: 36 has significant sequence homology with a phosphoglycerate kinase from S. cerevisiae. An amino acid part-sequence derived therefrom (corresponding to nucleotides 2 to 91 from SEQ ID NO: 36) with a part-sequence of the S. cerevisiae enzyme is depicted in FIG. 9. SEQ ID NO: 37 shows an N-terminally extended amino acid part-sequence.

The A. gossypii nucleic acid sequence found could thus be assigned the function of a phosphoglycerate kinase.

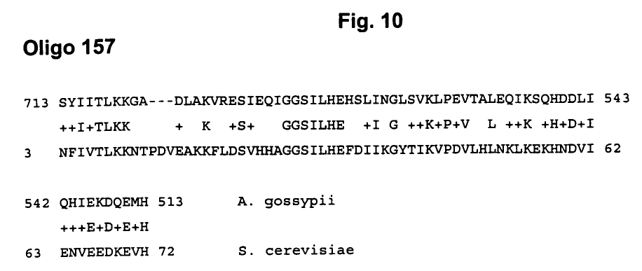

k) The amino acid sequence derived from the corresponding complementary strand to SEQ ID NO: 40 has significant sequence homology with a proteinase B inhibitor 2 from S. cerevisiae. An amino acid part-sequence derived therefrom (corresponding to nucleotides 713 to 513 from SEQ ID NO: 40) with a part-sequence of the S. cerevisiae enzyme is depicted in FIG. 10. SEQ ID NO: 41 shows an N-terminally extended amino acid part-sequence.

The A. gossypii nucleic acid sequence found could thus be assigned the function of a proteinase B inhibitor 2.

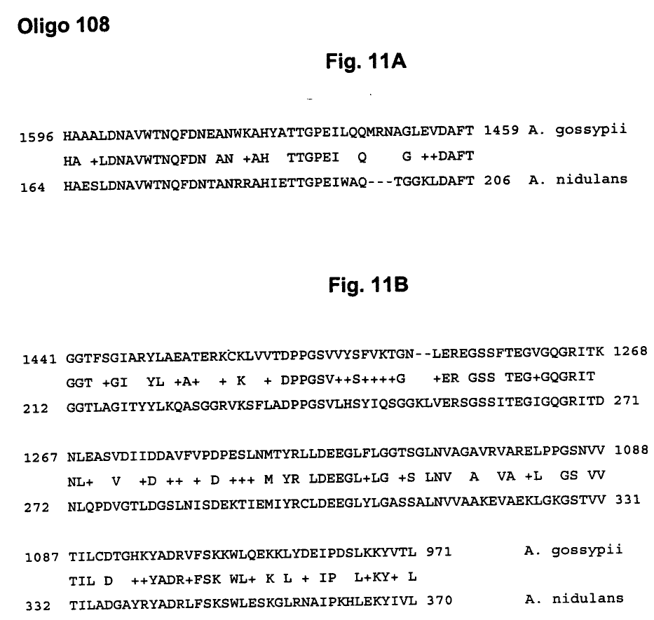

I) The amino acid sequence derived from the corresponding complementary strand to SEQ ID NO: 44 has significant sequence homology with a cysteine synthase from S. cerevisiae and A. nidulans. An amino acid part-sequence derived therefrom (corresponding to nucleotides 1596 to 1459 from SEQ ID NO: 44) with a part-sequence of the A. nidulans enzyme is depicted in FIG. 11 A. A further amino acid part-sequence derived therefrom (corresponding to nucleotides 1441 to 971 from SEQ ID NO: 44) with a part-sequence of the A. nidulans enzyme is depicted in FIG. 11B. SEQ ID NO: 45 and SEQ ID NO: 46 each show an N-terminally extended amino acid part-sequence.

The A. gossypii nucleic acid sequence found could thus be assigned the function of a cysteine synthase.

### EXAMPLE 6

Isolation of Full-Length DNA

a) Construction of an A. gossypigene library

High molecular weight cellular complete DNA from A. gossypii was prepared from a 2-day old 100 ml culture grown in a liquid MA2 medium (10 g of glucose, 10 g of peptone, 1 g of yeast extract, 0.3 g of myo-inositol ad 1000 ml). The mycelium was filtered off, washed twice with distilled H₂O, suspended in 10 ml of 1 M sorbitol, 20 mM EDTA, containing 20 mg of zymolyase 20T, and incubated at 27° C., shaking gently, for 30 to 60 min. The protoplast suspension was adjusted to 50 mM Tris-HCl, pH 7.5, 150 mM NaCl, 100 mM EDTA and 0.5% strength sodium dodecyl sulfate (SDS) and incubated at 65° C. for 20 min. After two extractions with phenovchloroform (1:1 vol/vol), the DNA was precipitated with isopropanol, suspended in TE buffer, treated with RNase, reprecipitated with isopropanol and suspended in TE.

An A. gossypii cosmid gene library was produced by binding genomic DNA which had been selected according to size and partially digested with Sau3A to the dephos-phorylated arms of the cosmid vector Super-Cos1 (Stratagene). The Super-Cos1 vector was opened between the two cos sites by digestion with Xbal and dephosphorylation with calf intestinal alkaline phosphatase (Boehringer), followed by opening of the cloning site with BamHl. The ligations were carried out in 20 μl, containing 2.5 μg of partially digested chromosomal DNA, 1 μg of Super-Cos1 vector arms, 40 mM Tris-HCl, pH 7.5, 10 mM MgCl₂, 1 mM dithiothreitol, 0.5 mM ATP and 2 Weiss units of T4-DNA ligase (Boehringer) at 15° C. overnight. The ligation products were packaged in vitro using the extracts and the protocol of Stratagene (Gigapack II Packaging Extract). The packaged material was used to infect E. coli NM554 (recA 13, araD139, Δ(ara,leu)7696, Δ(lac)17A, galU, galK, hsrR, rps(str^{r}), mcrA, mcrB) and distributed on LB plates containing ampicillin (50 μg/ml). Transformants containing an A. gossypii insert with an average length of 3045 kb were obtained.

b) Storage and screening of the cosmid gene library

In total, 4×10⁴ fresh single colonies were inoculated singly into wells of 96-well microtiter plates (Falcon, No. 3072) in 100 μl of LB medium, supplemented with the freezing medium (36 mM K₂HPO₄/13.2 mM KH₂PO₄, 1.7 mM sodium citrate, 0.4 mM MgSO₄, 6.8 mM (NH₄)₂SO₄, 4.4% (w/v) glycerol) and ampicillin (50 μg/ml), allowed to grow at 37° C. overnight with shaking, and frozen at −70° C. The plates were rapidly thawed and then duplicated in fresh medium using a 96-well replicator which had been sterilized in an ethanol bath with subsequent evaporation of the ethanol on a hot plate. Before the freezing and after the thawing (before any other measures) the plates were briefly shaken in a microtiter shaker (Infors) in order to ensure a homogeneous suspension of cells. A robotic system (Bio-Robotics) with which it is possible to transfer small amounts of liquid from 96 wells of a microtiter plate to nylon membrane (GeneScreen Plus, New England Nuclear) was used to place single clones on nylon membranes. After the culture had been transferred from the 96-well microtiter plates (1920 clones), the membranes were placed on the surface of LB agar with ampicillin (50 μg/ml) in 22×22 cm culture dishes (Nunc) and incubated at 37° C. ovemight. Before cell confluence was reached, the membranes were processed as described by Herrmann, B. G., Barlow, D. P. and Lehrach, H. (1987) in Cell 48, pp. 813-825, including as additional treatment after the first denaturation step a 5-minute exposure of the filters to vapors on a pad impregnated with denaturation solution on a boiling water bath.

The random hexamer primer method (Feinberg, A. P. and Vogelstein, B. (1983), Anal. iochem. 132, pp.6-13) was used to label double-stranded probes by uptake of [alpha-³²P]dCTP with high specific activity. The membranes were prehybridized and hybridized at 42° C. in 50% (vovvol) formamide, 600 mM sodium phosphate, pH 7.2, 1 mM EDTA, 10% dextran sulfate, 1 % SDS, and 10× Denhardt's solution, containing salmon sperm DNA (50 μg/ml) with ³²P-labeled probes (0.5-1×10⁶cpm/ml) for 6 to 12 h. Typically, washing steps were carried out at 55 to 65° C. in 13 to 30 mM NaCI, 1.5 to 3 mM sodium citrate, pH 6.3, 0.1 % SDS for about 1 h and the filters were autoradiographed at −70° C. with Kodak intensifying screens for 12 to 24 h. To date, individual membranes have been reused successfully more than 20 times. Between the autoradiographies, the filters were stripped by incubation at 95° C. in 2 mM Tris-HCI, pH 8.0, 0.2 mM EDTA, 0.1% SDS for 2×20 min.

c) Recovery of positive colonies from the stored gene library

Frozen bacterial cultures in microtiter wells were scraped out using sterile disposable lancets, and the material was streaked onto LB agar Petri dishes containing ampicillin (50 μg/mI). Single colonies were then used to inoculate liquid cultures to produce DNA by the alkaline lysis method (Birnboim, H. C. and Doly, J. (1979), Nucleic Acids Res. 7, pp.1513-1523).

d) Full-length DNA

It was possible as described above to identify clones which harbor an insert with the appropriate complete sequence. These clones had the internal names:

“Oligo 72v”. The insert comprising the complete sequence has a nucleic acid sequence as shown in SEQ ID NO: 4.

“Oligo 81 v”. The insert comprising the complete sequence has a nucleic acid sequence as shown in SEQ ID NO: 9. The protein encoded thereby preferably comprises at least one of the amino acid sequences as shown in SEQ ID NO: 10 and 11.

“Oligo 86v”. The insert comprising the complete sequence has a nucleic acid sequence as shown in SEQ ID NO: 14.

“Oligo 162v”. The insert comprising the complete sequence has a nucleic acid sequence as shown in SEQ ID NO: 18.

“Oligo 178v”. The insert comprising the complete sequence has a nucleic acid sequence as shown in SEQ ID NO: 22.

“Oligo 64v”. The insert comprising the complete sequence has a nucleic acid sequence as shown in SEQ ID NO: 26.

“Oligo 125v”. The insert comprising the complete sequence has a nucleic acid sequence as shown in SEQ ID NO: 30.

“Oligo 107v”. The insert comprising the complete sequence has a nucleic acid sequence as shown in SEQ ID NO: 34.

“Oligo 136v”. The insert comprising the complete sequence has a nucleic acid sequence as shown in SEQ ID NO: 38.

“Oligo 157v”. The insert comprising the complete sequence has a nucleic acid sequence as shown in SEQ ID NO: 42.

“Oligo 108v”. The insert comprising the complete sequence has a nucleic acid sequence as shown in SEQ ID NO: 47.

TABLE 1
Sequence survey

| SEQ | | | |
|---|---|---|---|
| ID NO: | Oligo | Description of the sequence | Sequence homology |
| 1 | 072 | DNA part-sequence | C1-Tetrahydrofolate synthase |
| 2 | 072 | Amino acid part-sequence derived from | from S. cerevisiae |
| | | the coding strand to SEQ ID NO: 1 | |
| 3 | 072 | Amino acid part-sequence derived from | |
| | | the coding strand to SEQ ID NO: 1 | |
| 4 | 072 | DNA full-length sequence | |
| 5 | 072 | Amino acid sequence corresponding to | |
| | | the coding region of SEQ ID NO: 4 from | |
| | | position 440 to 3253 | |
| 6 | 081 | DNA part-sequence | Argininosuccinate synthase from |
| 7 | 081 | Amino acid part-sequence derived from | S. cerevisiae |
| | | the complementary strand to SEQ ID | |
| | | NO: 6 | |
| 8 | 081 | Amino acid part-sequence derived from | |
| | | the complementary strand to SEQ ID | |
| | | NO: 6 | |
| 9 | 081 | DNA full-length sequence | |
| 10 | 081 | Amino acid sequence corresponding to | |
| | | the coding region of SEQ ID NO: 9 from | |
| | | position 716 to 1957 | |
| 11 | 081 | Amino acid sequence corresponding to | |
| | | the coding region of SEQ ID NO: 9 from | |
| | | position 2246 to 2839 | |
| 12 | 086 | DNA part-sequence | Phosphoribosylaminoimidazole- |
| 13 | 086 | Amino acid part-sequence derived from | succinocarboxamide synthase |
| | | the complementary strand to SEQ ID | from S. cerevisiae |
| | | NO: 12 | |
| 14 | 086 | DNA full-length sequence | |
| 15 | 086 | Amino acid sequence corresponding to | |
| | | the coding region of SEQ ID NO: 14 from | |
| | | position 587 to 1492 | |
| 16 | 162 | DNA part-sequence | Fumarate reductase from |
| 17 | 162 | Amino acid part-sequence derived from | S. cerevisiae |
| | | the coding strand to SEQ ID NO: 16 | |
| 18 | 162 | DNA full-length sequence | |
| 19 | 162 | Amino acid sequence corresponding to | |
| | | the coding region of SEQ ID NO: 18 from | |
| | | position 264 to 1772 | |
| 20 | 178 | DNA part-sequence | Phosphoenolpyruvate |
| 21 | 178 | Amino acid part-sequence derived from | carboxykinase from |
| | | the complementary strand to SEQ ID | S. cerevisiae |
| | | NO: 20 | |
| 22 | 178 | DNA full-length sequence | |
| 23 | 178 | Amino acid sequence corresponding to | |
| | | the coding region of SEQ ID NO: 22 from | |
| | | position 394 to 2016 | |
| 24 | 64 | DNA part-sequence | Uroporphyrinogen |
| 25 | 64 | Amino acid part-sequence derived from | decarboxylase from |
| | | the coding strand to SEQ ID NO: 24 | S. cerevisiae |
| 26 | 64 | DNA full-length sequence | |
| 27 | 64 | Amino acid sequence corresponding to | |
| | | the coding region of SEQ ID NO: 26 from | |
| | | position 612 to 1712 | |
| 28 | 125 | DNA part-sequence | Siroheme synthase from |
| 29 | 125 | Amino acid part-sequence derived from | S. cerevisiae |
| | | the complementary strand to SEQ ID | |
| | | NO: 28 | |
| 30 | 125 | DNA full-length sequence | |
| 31 | 125 | Amino acid sequence corresponding to | |
| | | the coding region of SEQ ID NO: 30 from | |
| | | position 275 to 2023 | |
| 32 | 107 | DNA part-sequence | Uroporphyrinogen III synthase |
| 33 | 107 | Amino acid part-sequence derived from | from S. cerevisiae |
| | | the coding strand to SEQ ID NO: 32 | |
| 34 | 107 | DNA full-length sequence | |
| 35 | 107 | Amino acid sequence corresponding to | |
| | | the coding region of SEQ ID NO: 34 from | |
| | | position 250 to 1035 | |
| 36 | 136 | DNA part-sequence | Phosphoglycerate kinase from |
| 37 | 136 | Amino acid part-sequence derived from | S. cerevisiae |
| | | the coding strand to SEQ ID NO: 36 | |
| 38 | 136 | DNA full-length sequence | |
| 39 | 136 | Amino acid sequence corresponding to | |
| | | the coding region of SEQ ID NO: 38 from | |
| | | position 447 to 1790 | |
| 40 | 157 | DNA part-sequence | Proteinase B inhibitor 2 from |
| 41 | 157 | Amino acid part-sequence derived from | S. cerevisiae |
| | | the complementary strand to SEQ ID | |
| | | NO: 40 | |
| 42 | 157 | DNA full-length sequence | |
| 43 | 157 | Amino acid sequence corresponding to | |
| | | the coding region of SEQ ID NO: 42 from | |
| | | position 809 to 1033 | |
| 44 | 108 | DNA part-sequence | Cysteine synthase from |
| 45 | 108 | Amino acid part-sequence derived from | S. cerevisiae and A. nidulans |
| | | the complementary strand to SEQ ID | |
| | | NO: 44 | |
| 46 | 108 | Amino acid part-sequence derived from | |
| | | the complementary strand to SEQ ID | |
| | | NO: 44 | |
| 47 | 108 | DNA full-length sequence | |
| 48 | 108 | Amino acid sequence corresponding to | |
| | | the coding region of SEQ ID NO: 47 from | |
| | | position 450 to 1517 | |

1. An isolated polynucleotide that can be isolated from Ashbya gossypii and that codes for a protein associated with metabolism of an organism.

2. The polynucleotide of claim 1, which has a structural or functional property of a protein selected from the group consisting of C1-tetrahydrofolate synthase, argininosuccinate synthase, phosphoribosylaminoimidazole succinocarboxamide synthase, fumarate reductase, phosphoenolpyruvate carboxykinase, uroporphyrinogen decarboxylase, siroheme synthase, uroporphyrinogen III synthase, phosphoglycerate kinase, proteinase B inhibitor 2 and cysteine synthase.

3. The polynucleotide of claim 1, comprising:
the nucleic acid sequence of SEQ ID NO: 1, 6, 12, 16, 20, 24, 28, 32, 36, 40 or 44;
a sequence complementary thereto; or
a sequence derived from said nucleic acid sequence or said sequence complementary thereto through degeneracy of the genetic code.

4. The polynucleotide of claim 1, which comprises a nucleic acid containing the sequence of SEQ ID NO: 4, 9, 14, 18, 22, 26, 30, 34, 38, 42 or 47, or a fragment thereof.

5. An oligonucleotide that hybridizes to the polynucleotide of claim 1.

6. An isolated polynucleotide that hybridizes to the oligonucleotide of claim 5, and codes for a gene product derived from a microorganism of the genus Ashbya or a functional equivalent thereof.

7. An isolated polypeptide encoded by the polynucleotide of claim 1 or a fragment thereof.

8. An expression cassette comprising the polynucleotide of claim 1 operatively linked to at least one regulatory sequence.

9. A recombinant vector comprising at least one expression cassette of claim 8.

10. A prokaryotic or eukaryotic host cell transformed with at least one vector of claim 9.

11. The host cell of claim 10, wherein functional expression of said protein is modulated.

12. A The host cell of claim 10, which is a microorganism of the genus Ashbya.

13. A method for microbiological production of vitamin B2 or a precursor or derivative thereof comprising expressing the polynucleotide of claim 1 in a microorganism.

14. A method for recombinant production of the polypeptide of claim 7 comprising expressing said polynucleotide in a microorganism.

15. A method for detecting an effector target for modulating microbiological production of vitamin B2 or a precursor or derivative thereof comprising treating a microorganism capable of the microbiological production of said vitamin B2 or the precursor or derivative thereof with an effector that interacts with a target wherein said target comprises the polypeptide of claim 7 or a nucleic acid that encodes said polypeptide and detecting said effector target.

16. A method for modulating microbiological production of vitamin B2 or a precursor or derivative thereof comprising treating a microorganism capable of the microbiological production of said vitamin B2 or the precursor or derivative thereof with an effector that interacts with a target wherein said target comprises the polypeptide of claim 7 or a nucleic acid that encodes said polypeptide.

17. An isolated effector selected from the group consisting of:
antibodies or antigen-binding fragments thereof that bind to the polypeptide of claim 7;
polypeptide ligands that are different from said antibodies or antigen-binding fragments and that interact with the polypeptide;
low molecular weight effectors that modulate a biological activity of a said polypeptide;
antisense nucleic acid sequences, catalytic RNA molecules and ribozymes which interact with a nucleic acid sequence that encodes said polypeptide; and
combinations and mixtures thereof.

18. A method for microbiological production of vitamin B2 or a precursor or derivative thereof comprising:
culturing the host cell of claim 10 under conditions favoring the production of vitamin B2 or the precursor or derivative thereof; and
isolating a desired product.

19. The method of claim 18, wherein the host cell is treated with an effector before or during culturing.

20. The method of claim 18, wherein the host cell is a microorganism of the genus Ashbya.

21. A method for modulating production of vitamin B2 or a precursor or derivative thereof in a microorganism of the genus Ashbya comprising treating said microorganism with the polynucleotide of claim 1.

22. A method for modulating production of vitamin B2 or a precursor or derivative thereof in a microorganism of the genus Ashbya comprising treating said microorganism with the polypeptide of claim 7 and hereby modulating production as desired.

23. A method for modulating a metabolic function of a microorganism of the genus Ashbya comprising culturing said microorganism for microbiological production of vitamin B2 or a precursor or derivative thereof with the polynucleotide of claim 1 or with a polypeptide encoded by said polynucleotide.

24. The host cell of claim 12, which has an improved metabolic regulation or modified metabolic function as compared with an untransformed microorganism, wherein said improved metabolic regulation or modified metabolic function provides for an increased production of vitamin B2 or a precursor or derivative thereof.

25. The polynucleotide of claim 1, wherein the organism is A. gossypii, S. cerevisiae or A. nidulans.

26. The polynucleotide of claim 1, wherein the protein is associated with synthesis or regulation of metabolic enzymes of the organism.

27. The polynucleotide of claim 2, wherein the protein is derived from a microorganism of A. gossypii, S. cerevisiae or A. nidulans.

28. The oligonucleotide of claim 5, wherein hybridization is under stringent conditions.

29. The polynucleotide of claim 6, wherein hybridization is under stringent conditions.

30. An isolated polypeptide or fragment thereof encoded by the polynucleotide of claim 6.

31. An isolated polypeptide or fragment thereof which has an amino acid sequence that comprises at least ten consecutive amino acid residues of SEQ ID NO:2, 3, 5, 7, 8, 10, 11, 13, 15, 17, 19, 21, 23, 25, 27, 29, 31, 33, 35, 37, 39, 41, 43, 45, 46 or 48; or a functional equivalent thereof.

32. The polypeptide of claim 31, which has an activity comparable with a protein selected from the group consisting of C1-tetrahydrofolate synthase, argininosuccinate synthasephosphoribosylaminoimidazole succinocarboxamide synthase, fumarate reductase, phosphoenolpyruvate carboxykinase, uroporphyrinogen decarboxylase, siroheme synthase, uroporphyrinogen III synthase, phosphoglycerate kinase, proteinase B inhibitor 2 and cysteine synthase.

33. The polypeptide of claim 32, wherein the protein is derived from a microorganism of A. gossypii, S. cerevisiae, or A. nidulans.

34. The host cell of claim 10, wherein biological activity of said protein is reduced or increased.

35. The method of claim 11, wherein modulating comprises an increase or decrease in the functional expression of said protein.

36. The method of claim 13, wherein expressing said polypeptide results in an improved production of vitamin B2 or a precursor or derivative thereof by said microorganism.

37. The method of claim 36, wherein the improved production comprises an increased yield, production or efficiency of production by said microorganism.

38. The method of claim 15, wherein detecting validates said effector target.

39. The method of claim 15, where the effector binds to said target.

40. The method of claim 15, further comprising isolating said target.

41. The method of claim 19, wherein the effector is selected from the group consisting of:
antibodies or antigen-binding fragments thereof that bind to a polypeptide associated with A. gossypii metabolism;
polypeptide ligands that are different from said antibodies or antigen-binding fragments and that interact with said polypeptide;
low molecular weight effectors that modulate a biological activity of said polypeptide;
antisense nucleic acid sequences, catalytic RNA molecules and ribozymes which interact with a nucleic acid sequence that encodes said polypeptide; and
combinations and mixtures thereof.

42. The method of claim 21, wherein modulating comprises an increase in rate or amount of the vitamin B2 or the precursor or derivative thereof produced by said microorganism.

43. The method of claim 22, wherein modulating comprises an increase in rate or amount of the vitamin B2 or the precursor or derivative thereof produced by said microorganism.

44. A recombinant cell with an improved metabolic regulation or modified metabolic function that provides for an increased production of vitamin B2, or a precursor or derivative thereof, as compared with a non-recombinant cell.

45. The recombinant cell of claim 37, which is A. gossypii, S. cerevisiae or A. nidulans.
